(19) United States
(12) Patent Application Publication
(10) Pub. No.: US 2007/0224174 A1
(43) Pub. Date: Sep. 27, 2007

(54) NATURAL KILLER CELL COMPOSITIONS AND METHOD FOR PRODUCTION OF THE SAME

(75) Inventors: Hyung Sik Kang, Gwangju (KR); Sang Hyun Kim, Seoul (KR); Eun Mi Kim, Gwangju (KR); Eun Hee Lee, Gwangju (KR); Hee Jung Yun, Hampyeong-gun (KR); Chang Bo Ko, Gongju-si (KR); Young Jin Heo, Seoul (KR); Woo Young Shim, Incheon (KR); Seong Beom Heo, Seoul (KR); Ji Yeon Yoon, Seoul (KR)

Correspondence Address: KNOBBE MARTENS OLSON & BEAR LLP, 2040 MAIN STREET, FOURTEENTH FLOOR, IRVINE, CA, 92614, US

(73) Assignees: INDUSTRY FOUNDATION OF CHONNAM NATIONAL UNIVERSITY, GWANGJU (KR); GOOD CELL LIFE, INC., SEOUL (KR)

(21) Appl. No.: 11/678,563

(22) Filed: Feb. 23, 2007

Related U.S. Application Data
(63) Continuation-in-part of application No. PCT/KR06/03627, filed on Sep. 12, 2006.

(30) Foreign Application Priority Data
Sep. 12, 2005 (KR) 10-2005-0084896
Sep. 12, 2005 (KR) 10-2005-0084897
Sep. 12, 2005 (KR) 10-2005-0084898
Jul. 26, 2006 (KR) 10-2006-0070390

(51) Int. Cl. A61K 35/14; A61K 38/20; C12N 5/08; A61K 39/395

(52) U.S. Cl. 424/93.7; 435/372; 424/85.2; 424/144.1

(57) ABSTRACT

The subject matter of the present invention relates to a ligand of Axl receptor tyrosine kinase used to induce the differentiation from precursor natural killer cell to mature natural killer cell. In addition, it relates to a process for producing mature natural killer cell comprising treating hematopoietic stem cells with interleukin-7, stem cell factor and Flt3L to differentiate into precursor natural killer cells, and treating the resulting precursor natural killer cells with at least one ligand of Axl receptor tyrosine kinase to produce mature natural killer cells.

### CROSS-REFERENCE TO RELATED APPLICATIONS

This application is a continuation-in-part application under 35 U.S.C. § 365(c) of International Application No. PCT/KR2006/003627, filed Sep. 12, 2005, designating the United States. International Application No. PCT/KR2006/003627 has not yet been published. This application further claims for the benefit of the earlier filing dates under 35 U.S.C. § 365(b) of Korean Patent Application No. 10-2005-0084896, filed Sep. 12, 2005, Korean Patent Application No. 10-2005-0084897, filed Sep. 12, 2005, Korean Patent Application No. 10-2005-0084898, filed Sep. 12, 2005, and Korean Patent Application No. 10-2006-0070390, filed Jul. 26, 2006. This application incorporates herein by reference the International Application No. PCT/KR2006/003627, Korean Patent Application No. 10-2005-0084896, Korean Patent Application No. 10-2005-0084897, Korean Patent Application No. 10-2005-0084898, and Korean Patent Application No. 10-2006-0070390 in their entirety.

### BACKGROUND

1. Field of the Invention

The present invention relates to a ligand of Axl receptor tyrosine kinase used to induce the differentiation from precursor natural killer cell to mature natural killer cell.

2. Description of the Related Technolgy

The modern-day approach to cancer management is a multidisciplinary one, consisting primarily of surgery, radiation therapy and chemotherapy, in varying combinations. However, these treatments function with temporary effect and have problems related to inducement of therapeutic resistance, recurrence of cancer, or adverse physiological effects. Research is currently underway to develop immunotherapies such as anticancer drugs and diagnostic reagents which can modulate immunological reaction, but this development is still in its infancy.

Natural killer cells (NK cells) are lymphocytes of innate immunity that remove pathogenic, cancerous, and allogeneic cells. NK cells mediate adaptive immunity by secreting cytokines such as interferon-γ tumor necrosis factor-alpha, and IL-12; they also have the ability to specifically control cancer and retain this “memory” for protection against future attacks. Previous cancer treatments utilizing NK cells have involved the enhancement of immune response against cancer cells by activating NK cells with interleukin-2. However, this therapy is known to have several problems, including adverse effects and differences in efficacy, tolerance and persistence among individuals.

NK cells are effective for clinical treatment due to the ability of NK cells to treat cancer, incurable virus infection, etc. In order to increase the cancer destroying effect of NK cells, it is useful to conduct treatment with a large amount of NK cells with high activity. It is estimated that approximately more than ten billion (10¹⁰) NK cells would be useful for clinical treatment. However, NK cells are very difficult to amplify. Various methods have been employed but only several tens of expansion have been achieved by using IL-2 or IL-15. However, it is known that expansion on the order of several hundred-fold is possible if cancer cells from various patients are mixed with the NK cells. According to a recent method for amplifying NK cells published in 2005, but which is not an admission of prior art, an amplification of 1000-fold was achieved when cancer cells from other patients to which two genes were added were mixed with the NK cells. However, this and other methods are still under study. For the amplification of at least several hundred-fold, it is known in the art to culture NK cells in admixture with cancer cells from different people. However, there are problems of safety and ethical concerns for mixing cancer cells or using genes from other people.

### SUMMARY

One aspect of the invention provides a composition comprising: a precursor natural killer cell comprising Axl receptor; and a ligand that is not naturally occurring in the precursor natural killer cell and that is configured to form a complex with the Axl receptor, wherein the complex, when formed, is configured to induce the precursor natural killer cell to differentiate into a mature natural killer cell.

In some embodiments of the foregoing composition, the ligand can be at least one selected from the group consisting of: γ-carboxylated Gas6 protein, γ-carboxylated Gas6 protein homologues, and fragments of γ-carboxylated Gas6 protein, fragments of γ-carboxylated Gas6 protein homologues, and an antibody configured to bind Axl receptor.

In some embodiments, the foregoing composition can further comprise a human stromal cell.

In some embodiments, the foregoing composition can further comprise a cell configured to express γ-carboxylated Gas6 protein, γ-carboxylated Gas6 protein homologues, or fragments of the foregoing. In a further embodiment of the foregoing composition, the cell can be a cell from a cloned cell line.

In some embodiments, the foregoing composition can further comprise a mature natural killer cell, wherein the mature natural killer cell is either activated or inactivated to target a cancer cell.

In some embodiments, the foregoing composition can further comprise a hematopoietic stem cell.

In another aspect of the invention, a composition is provided comprising: a mature natural killer cell comprising an Axl receptor; and a ligand that is not naturally occurring in the mature natural killer cell, wherein the ligand and the Axl receptor are in the form of a complex.

In some embodiments, the foregoing composition can further comprise a precursor natural killer cell.

Another aspect of the invention provides a method to produce the foregoing composition, comprising: providing a cell culture; providing a precursor natural killer cell in the culture, wherein the precursor natural killer cell comprises an Axl receptor; and providing in the culture a ligand that is not naturally occurring in the precursor natural killer cell such that the ligand and the precursor natural killer cell contact with each other and such that the ligand and the Axl receptor form a complex, wherein the complex, when formed, induces the precursor natural killer cell to differentiate into a mature natural killer cell.

In some embodiments, the foregoing method further comprises separating the mature natural killer cell from the culture. In further embodiments, the method further comprises culturing the mature natural killer cell in interleukin-2. In still further embodiments of the foregoing method, the concentration of interleukin-2 is from 8 to 15 ng/mL.

In some embodiments of the foregoing method, providing the precursor natural killer cell comprises: providing a population of hematopoietic stem cells; and differentiating at least a part of the population of hematopoietic stem cells into a population comprising precursor natural killer cells.

In another aspect of the invention, a method is provided to treat a patient in need of mature natural killer cells, comprising: providing a composition comprising a mature natural killer cell comprising an Axl receptor and a ligand that is not naturally occurring in the mature natural killer cell, wherein the ligand and the Axl receptor are in the form of a complex; and administering the composition to the patient in an amount that is effective to treat the patient.

In some embodiments of the foregoing method, the composition is administered in an amount that is effective for the treatment of cancer in the patient.

Another aspect of the invention provides a method to treat cancer, comprising: obtaining a population of hematopoietic stem cells; differentiating at least a part of the population of hematopoietic stem cells into a population comprising precursor natural killer cells, wherein the precursor natural killer cell comprises an Axl receptor; contacting at least a part of the population comprising precursor natural killer cells with a ligand that is not naturally occurring in the precursor natural killer cells, thereby differentiating at least part of the precursor natural killer cells into mature natural killer cells; and administering at least part of the mature natural killer cells to a patient.

In some embodiments, the foregoing method is provided, wherein the population of hematopoietic stem cells is obtained from at least one human blood source selected from the group consisting of: bone marrow, peripheral blood, and umbilical cord blood.

In some embodiments, the foregoing method is provided, wherein the population of hematopoietic stem cells is obtained from the patient.

In some embodiments, the foregoing method is provided, additionally comprising separation of the at least part of the mature natural killer cells from non-mature natural killer cells.

In some embodiments, the foregoing method is provided, wherein the ligand is at least one selected from the group consisting of: γ-carboxylated Gas6 protein, γ-carboxylated Gas6 protein homologues, fragments of γ-carboxylated Gas6 protein, fragments of γ-carboxylated Gas6 protein homologues, and an antibody that is configured to bind Axl receptor.

### BRIEF DESCRIPTION OF THE DRAWINGS

FIG. 1 shows a flow diagram illustrating the isolation and differentiation of murine bone marrow-derived hematopoietic stem cells into mature natural killer cells (BM: bone marrow; HSC: hematopoietic stem cells; pNK: precursor natural killer cells; mNK: mature natural killer cells).

FIG. 2 shows purity data of differentiation-staged cells analyzed by FACS which were developed from HSCs isolated from murine BM according to the prior art method into mNK cells.

FIG. 3 shows an electrophoretic analysis following RT-PCR of specific gene expression in murine BM-derived HSCs, pNK cells differentiated from HSCs, and mNK cells developed from the pNK cells in the absence and presence of OP9 stromal cells.

FIG. 4 shows purity data of mNK cells analyzed by FACS which were differentiated by culturing pNK cells either alone or in co-culture with OP9 stromal cells.

FIG. 5 shows the schematic diagram of the steps in SAGE of differentiation-staged cells during the differentiation from HSCs to mNK cells.

FIG. 6 shows the comparison of electrophoresis and SAGE analyses of Axl in differentiation-staged cells during the differentiation from murine HSCs to mNK cells, both being consistent.

FIG. 7 shows the effect of using polyclonal antibody directed against Axl according to the subject matter of the present invention on the differentiation from murine pNK cells to mNK cells, analyzed by FACS.

FIG. 8 shows the effect of using polyclonal antibody directed against Axl according to the subject matter of the present invention on the differentiation from murine pNK cells to mNK cells, analyzed by electrophoresis following RT-PCR.

FIG. 9 shows the effect of using polyclonal antibody directed against Axl according to the subject matter of the present invention in combination with a low concentration of IL-15 in the absence of stromal cells on the differentiation from murine pNK cells to mNK cells, analyzed by FACS.

FIG. 10 shows the effect of using polyclonal antibody directed against Axl according to the subject matter of the present invention in combination with a low concentration of IL-15 in the absence of stromal cells on the differentiation from murine pNK cells to mNK cells, analyzed by electrophoresis following RT-PCR.

FIG. 11 shows the effect of using polyclonal antibody directed against Axl according to the subject matter of the present invention on the production of interferon-γ by mNK cells.

FIG. 12 shows the effect of using polyclonal antibody directed against Axl according to the subject matter of the present invention on the proliferation of pNK cells.

FIG. 13 shows the effect of using murine recombinant Gas6 according to the subject matter of the present invention on the differentiation of murine NK cells.

FIG. 14 shows the effect of warfarin on the differentiation of murine NK cells.

FIG. 15 shows the electrophoretic analysis of the recombinant vector containing cloned murine Gas6 cDNA according to the subject matter of the present invention.

FIG. 16 shows the electrophoretic analysis of the recombinant murine Gas6 expression vector of one embodiment of the present invention.

FIG. 17 shows the electrophoretic analysis of the cloning of murine Gas6 cDNA in the constructed expression vector.

FIG. 18 shows the electrophoretic analysis of the Gas6 expression in murine Gas6 transfectant according to one embodiment of the present invention.

FIG. 19 shows the effect of the murine Gas6 transfectant according to the subject matter of the present invention on the differentiation from murine pNK cells to mNK cells, analyzed by FACS.

FIG. 20 shows the effect of the murine Gas6 transfectant according to the subject matter of the present invention on the differentiation from murine pNK cells to mNK cells, analyzed by electrophoresis following RT-PCR.

FIG. 21 shows the effect of the murine Gas6 transfectant according to the subject matter of the present invention on the production of interferon-γ by mNK cells.

FIG. 22 shows the effect of the murine native Gas6 on the proliferation of pNK cells.

FIG. 23 shows the electrophoretic analysis of the cloning of murine Axl cDNA and the direction of the murine Axl cDNA cloned in retrovirus vector pLXSN.

FIG. 24 shows the electrophoretic analysis of the murine Axl-Fc expression vector according to the subject matter of the present invention.

FIG. 25 shows the effect of the murine Axl-Fc fusion protein according to the subject matter of the present invention on the differentiation of NK cells.

FIG. 26 shows the vector system of the subject matter of the present invention based on self-inactivating Lenti virus.

FIG. 27 shows the construction of double-promoter siRNA cassette and siRNA template oligomer used in the subject matter of the present invention.

FIG. 28 shows the electrophoretic analysis of the cloning of Axl siRNA according to the subject matter of the present invention.

FIG. 29 shows the fluorescence and FACS analyses of the level of infection by pFIV-U6/H1-GFP virus in 293T cells.

FIG. 30 shows the FACS analysis of the level of infection by pFIV-U6/H1-GFP virus in murine HSCs and pNK cells.

FIG. 31 shows the effect of Axl siRNA according to the subject matter of the present invention on the differentiation of murine mNK cells, analyzed by FACS.

FIG. 32 shows the tumorigenesis-inhibiting effect of NK cells differentiated by Axl polyclonal antibody according to the subject matter of the present invention in an animal model.

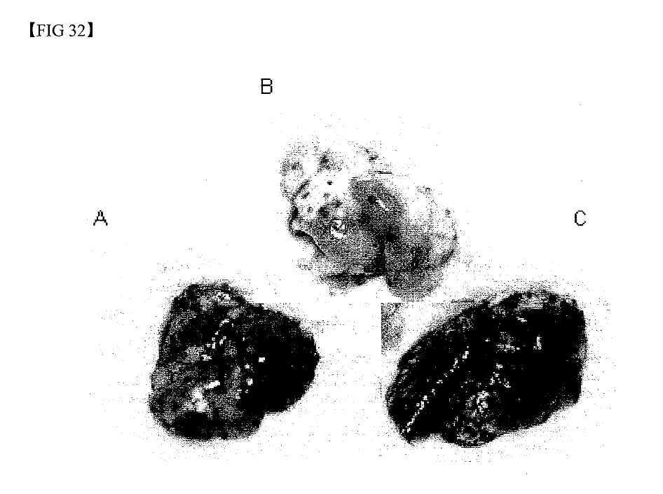

FIG. 33 shows the cancer cell-killing effect of NK cells differentiated by Axl polyclonal antibody according to the subject matter of the present invention in an animal model.

FIG. 34 shows survival rate of mouse with induced cancer to which NK cells differentiated by Axl polyclonal antibody according to the subject matter of the present invention were injected.

FIG. 35 shows a recombinant expression vector pET-hAxl/ECD of one embodiment of the present invention producing human Axl protein.

FIG. 36 shows a recombinant expression vector phGas6 of one embodiment of the present invention producing human Gas6 protein.

FIG. 37 shows purity data of mNK cells by FACS which were obtained by separating HSCs from human umbilical cord blood, differentiating them into pNK cells by treating with SCF, Flt3-L, and IL-7, and then differentiating the resulting pNK cells into mNK cells by treating with IL-15.

FIG. 38 shows expression data for perforin and granzyme of the mNK cells analyzed by electrophoresis following RT-PCR, which were obtained by separating HSCs from human umbilical cord blood, differentiating them into pNK cells by treating with SCF, Flt3-L, and IL-7, and differentiating the resulting pNK cells into mNK cells by treating with IL-15.

FIG. 39 shows the cancer cell-killing effect of mNK cells which were obtained by separating HSCs from human umbilical cord blood, differentiating them into pNK cells by treating with SCF, Flt3-L, and IL-7, and then differentiating the resulting pNK cells into mNK cells by treating with IL-15 in cell level experiment.

FIG. 40 shows purity data of mNK cells which were obtained by separating HSCs from human umbilical cord blood, differentiating them into pNK cells, and differentiating the resulting pNK cells into mNK cells by treating with a polyclonal antibody against Axl protein, analyzed by FACS.

FIG. 41 shows purity data of mNK cells which were obtained by separating HSCs from human peripheral blood, differentiating them into pNK cells, and differentiating the resulting pNK cells into mNK cells by treating with a polyclonal antibody directed against Axl protein, analyzed by FACS.

### DETAILED DESCRIPTION OF EMBODIMENTS

An embodiment of the present invention is to provide mature NK cells in large quantity by inducing the differentiation from precursor NK cells to mature NK cells.

Another embodiment of the present invention is to provide a substance which induces the differentiation from precursor NK cells to mature NK cells.

Yet another embodiment of the present invention is to provide activated mature NK cells which are obtained from mature NK cells by treating with low dose of IL-2.

Yet another embodiment of the present invention is to provide an immune cell therapeutic composition containing mature NK cells differentiated and activated in accordance with the present invention.

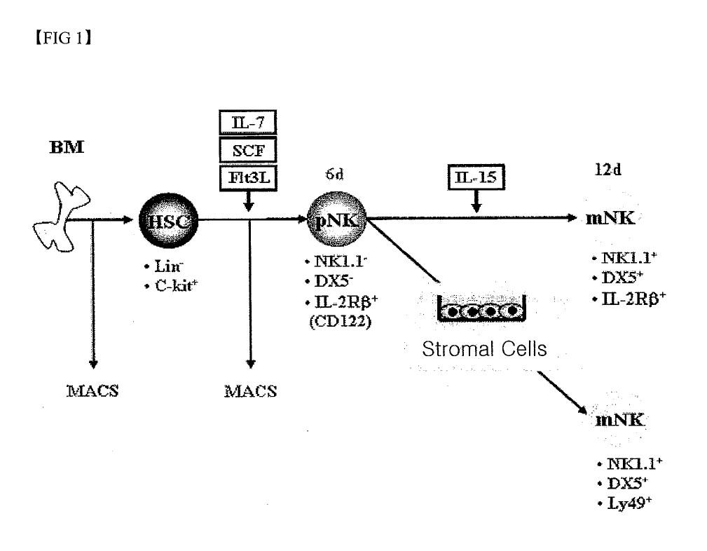

The inventors investigated the differentiation process of hematopoietic stem cells (HSCs) into mature natural killer (mNK) cells (FIG. 1). The inventors found that the Axl gene is involved in the differentiation process. Axl (p140) is a tyrosine receptor phosphorylase belonging to a family of Sky and Eyk. Other members that belong to the family of Sky and Eyk include Rse (Sky, Brt, Tif, Dtk, Tyro3) and Mer (Eyk, Nyk, Tyro12). It is known that Axl, Rse and Mer are expressed in most of tissues, but their function remains unknown.

Gas6 is known as a ligand of Axl protein and is a vitamin K-dependent potentiating factor believed to be involved with Axl-related cell response, including migration, growth, and differentiation. It has been reported that Axl protein is expressed in fibroblasts, myeloid progenitors, macrophages, neural tissues, follicles, and skeletal muscle but not in lymphocytes. Although Axl and Gas6 are known to regulate homeostasis of antigen presenting cells and growth of hematopoietic cells, the regulation of the development and function of NK cells by Axl and Gas6 is not known. The inventors of the present invention found that Axl protein plays an essential role in differentiation of pNK cells into mNK cells. Based on the unexpected discovery of the function of Axl protein as a differentiation regulator, the inventors isolated hematopoietic cells from mouse bone marrow and then established a system to differentiate the hematopoietic cells into mNK cells using Axl antibody, Gas6 protein and homologues thereof, or combinations of both Axl antibody and Gas6 protein or protein homologues. Further, by modifying the differentiation system and applying it to hematopoietic cells isolated from human peripheral blood, bone marrow or umbilical cord blood, mNK cells with enhanced activity were successfully produced in a large amount.

Following the result described above, in one aspect of the present invention, a ligand for Axl protein which induces the differentiation of hematopoietic cells into mNK cells is provided. The ligand can be, for example, at least one of the following: an antibody against Axl protein, Gas6 protein and protein S. In some embodiments, the ligand can be any combination of polypeptides or compounds that specifically bind Axl protein. The term “differentiation” means a phenomenon that a relatively simple system is divided into at least two partial systems which are qualitatively different from the original system. In other words, the differentiation means a phenomenon in which a structure or function becomes specialized. In mNK cells, the term ‘maturation’ means that NK cells become developed and exert their intrinsic cellular functions; for example, recognition of and direct killing of cancer cells. Maturation of NK cells can be confirmed by detection of an expressed substance or marker that is well-known in the art. A typical mouse marker includes, for example, NK1.1, CD122, LY49 Family (Ly49A, Ly49C, Ly49D, Ly49E, Ly49F, Ly49G, Ly49H, and Ly49I), or NKG2A/C/E. A typical human marker includes, for example, NKG2A, NKG2D, NKp30, NKp44, NKp46, CD56, and CD161. The expression, detection and function of these markers is well known in the art.

As used herein, the term “ligand” can include a polypeptide, protein, amino acid sequence or compound which specifically binds Axl receptor and thus induces an agonistic response of the receptor protein. The ligand can be at least one, two, three or more different polypeptides, proteins, amino acid sequence, compound, or any combinations thereof that bind Axl receptor. The ligand can be a polypeptide that is at least 5, 7, 10, 20, 30, 50, 100, 150, 200, 250, 300, 400 or 500 amino acids in length. The ligand can be a polypeptide that is, for example, a recombinantly expressed, substantially purified sequence, or can be synthetically prepared. The ligand can be prepared biologically in a host cell.

The term “polypeptide” as used herein generally refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

In some embodiments of the present invention, the full length ligand for Axl receptor can be used. In additional embodiments, a fragment of the ligand is used. The fragment of the ligand for Axl receptor can be, for example, at least about 5, 7, 10, 20, 30, 50, 100, 150, 200, 250, 283, 285, 300, or 303 amino acids of the full length ligand. In some embodiments, fragment can be, for example, as little as 10%, 15%, 20%, 25%, 30%, 35%, 40%, 45%, 50%, 55%, 60%, 65%, 70%, 75%, 80% or 85% of the full length ligand for Axl receptor.

In some embodiments, the ligand that binds Axl receptor can be a homologue of a known ligand. A homologue includes a polypeptide that is at least about 60% identical to a known ligand of Axl receptor, including but not limited to about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100%. In further embodiments, the polypeptide is at least about 75% identical to a known ligand of Axl receptor. In further embodiments, the polypeptide is functional Gas6, Protein S, or antibody specific for binding Axl receptor. In some embodiments, by functional is meant that the homologue has the function or activity of Gas6, Protein S, or an antibody specific for binding Axl receptor. To the extent that it can generate a complex that can effectively induce the precursor NK cell to differentiate into a mature NK cell, a homologue or fragment of a ligand the binds Axl receptor can be used in some embodiments of the present invention.

Further embodiments are polynucleotides that encode ligands of Axl protein. In some embodiments, the polynucleotide is a native sequence for Gas6 or Protein S. In further embodiments, the polynucleotide is a derived sequence in which the codon usage for E. coli or a suitable host organism is used to express a polypeptide that is at least 72.5% identical to the native sequence of Gas6 or Protein S. In further embodiments, the polynucleotide is at least about 65% identical to the polynucleotide sequence from native Gas6 or Protein S. In further embodiments, the polynucleotide is at least about 70% identical, including but not limited to: 75%, 77%, 80%, 85%, 90%, 95%, 97.5%, and 99% to the native sequence for Gas6 or Protein S. In further embodiments, the polynucleotide sequence encodes an active or functional ligand for Axl protein, such as, for example, Gas6 or Protein S, as described above.

In another aspect of the present invention, a method of producing mNK cells is provided, characterized in that (i) hematopoietic cells are treated with IL-7, SCF and Flt3L to differentiate into precursor NK cells, and (ii) the resulting precursor NK cells are treated with a ligand of Axl protein to differentiate into mNK cells, thereby obtaining mNK cells.

An embodiment of the present invention provides a method to produce human mNK cells characterized in that hematopoietic cells are isolated from human peripheral blood, bone marrow or umbilical cord blood.

In another embodiment of the present invention, a method of producing human mNK cells is provided, characterized in that a ligand of Axl protein is selected from a group consisting of an antibody against Axl protein, human γ-carboxylated Gas 6 and mixtures thereof.

In another embodiment of the present invention, a method of producing human mNK cells is provided, characterized in that precursor mNK cells are treated with the ligand in the presence of human stromal cell.

In another aspect of the present invention, a method of producing activated mNK cells is provided, characterized in that (i) treating hematopoietic cells with IL-7, SCF and Flt3L to differentiate them into precursor mNK cells, (ii) treating the resulting precursor mNK cells with the ligand of Axl protein to differentiate them into mNK cells, and therefore obtaining mNK cells, and (iii) activating the differentiated mNK cells with the treatment of IL-2. In a further embodiment of the present invention, the method is characterized in that the differentiated mNK cells are treated with about 8 to 15 ng/ml of IL-2.

In another aspect of the present invention, an immune cell therapy composition is provided, characterized in that it comprises the activated mNK cells prepared by the process of the present invention. In the text of the present invention, it is understood that the immune cell therapy composition can be used as various anticancer agent or immunostimulant, etc. The term “activation” in the activated mNK cells means that after being stimulated by IL-2 mNK cells show a substantial cytotoxic effect.

In another aspect of the present invention, an autoimmune cell therapy is provided, characterized in that, hematopoietic cells are isolated from blood and/or bone marrow of a patient to be treated, the resulting hematopoietic cells are treated with IL-7, SCF and Flt3L to differentiate them into precursor mNK cells, the resulting precursor mNK cells are treated with the ligand for Axl protein to differentiate them into mNK cells, the resulting mNK cells are activated with the treatment of IL-2, and the resulting activated mNK cells are introduced back to the patient to be treated. As used herein, “autoimmune cell therapy” means that the patient's own immune cells isolated from the body of the patient or immune cells obtained in vitro via differentiation using the patient's own hematopoietic cells, which can selectively destroy cancer cells, are amplified or strengthened via in vitro culture, and then the cells are re-introduced into the patient's own body in order to treat cancer.

In another aspect of the present invention, an alloimmune cell therapy is provided, characterized in that, hematopoietic cells are isolated from human alloumbilical cord blood, donated blood and/or bone marrow, the resulting hematopoietic cells are treated with IL-7, SCF and Flt3L to differentiate them into precursor mNK cells, the resulting precursor mNK cells are treated with the ligand for Axl protein in the presence of a human stromal cell to differentiate into mNK cells, the resulting mNK cells are activated with the treatment of IL-2, and the resulting activated mNK cells are introduced to the patient to be treated. As used herein, “alloimmune cell therapy” means that fetal umbilical cord blood is differentiated into immune cells by using adult stem cells in vitro, thus the immune cells which can selectively destroy cancer cells are amplified or strengthened and are introduced to a patient's body to treat cancer.

An “effective amount” of a composition comprising a mNK cell will depend, for example, upon the therapeutic objectives, the route of administration, the type of compound employed, and the condition of the patient. Accordingly, the practitioner can titer the dosage and modify the route of administration as required to obtain the optimal therapeutic effect. Typically, the clinician will administer the compound until a dosage is reached that achieves the desired effect. The progress of this therapy is easily monitored by conventional assays.

Natural killer (NK) cells are white blood lymphocytes involved in the innate immune system that have diverse biological functions including recognition and destruction of certain microbial infections and neoplasms (Moretta, A., Bottino, C., Mingari, M. C., Biassoni, R. and Moretta, L., Nat. Immunol., 3, 6, 2002, which is incorporated herein by reference in its entirety). Their morphological characteristic shows a large granular lymphocyte (LGL)-like morphology based on the presence of densely staining azurophilic granules in their cytoplasm. They comprise approximately 10 to 20% of the population in normal peripheral blood lymphocytes, approximately 15 to 25% of the population in liver lymphocytes, and approximately 1 to 5% of the population in spleen lymphocytes. Resting NK cells circulate in the blood, but following activation by cytokines, they are capable of extravasation and infiltration into most tissues that contain pathogen-infected or malignant cells (Colucci, F., Di Santo, J. P. and Leibson, P. J., Nat. Immunol., 3, 807, 2002; Kelly J. M., Darcy P. K., Markby J. L., Godfrey D. I., Takeda K., Yagita H., Smyth M. J., Nat. Immunol., 3, 83, 2002; Shi F. D., Wang H. B., Li H., Hong S., Taniguchi M., Link H., Van Kaer L., Ljunggren H. G., Nat. Immunol., 1, 245, 2000; Korsgren M., Persson C. G., Sundler F., Bjerke T., Hansson T., Chambers B. J., Hong S., Van Kaer L., Ljunggren H. G., Korsgren O., J. Exp. Med., 189, 553, 1999, each of which is incorporated herein by reference in its entirety). In general, the phenotype of NK cells is characterized by the expression of the CD56 and CD16 (in human), NKR-P1C(NK1.1 in mouse, CD161 in human), DX5, Ly49 (in mouse; these are restricted to certain mouse strains) surface antigen, and the lack of CD3. The majority (comprising 90% of total NK cells) of human NK cells have low-density expression of CD56 (CD56^{dim}, more cytotoxic) and express high levels of Fcg receptor III (FcgRIII, CD16), whereas approximately 10% of NK cells are CD56^{bright}CD16^{dim} or CD56^{bright}CD16⁻ (Schattner, A. and Duggan, D. B., Arthritis Rheum., 27, 1072, 1984, which is incorporated herein by reference in its entirety). Unlike other immune cells, NK cells can destroy virus-infected cells and tumor cells, etc. in the absence of prior sensitization. NK cells play a central role in early host defense via interaction of activating receptors with their ligands.

NK cells have an ability to discriminate between normal cells and cells lacking the expression of major histocompatibility complex (MHC) class I molecules due to the recognition by NK inhibitory receptors that are specific for MHC class I molecules. The functions of NK cells are regulated by a balance between activating receptors and inhibitory receptors that interact with their ligand such as MHC class I or MHC class I-related molecules (non-classical MHC class I) on the target cells (Rajaram, N., Tatake, R. J., Advani, S. H. and Gangal, S. G., Br. J. Cancer, 62, 205, 1990, which is incorporated herein by reference in its entirety). These receptors are divided into two structural families: the immunoglobulin superfamily (leukocyte inhibitory receptors, killer cell Ig-like receptors (KIR, CD158) and the C-type lectin-like family (NKG2D, CD94/NKG2, lymphocyte antigen 49 (LY49)). NK cells also produce several cytokines, such as interferon-γ (IFN-γ) and tumor-necrosis factor-α (TNF-α), following interaction of cell-surface receptors with their ligand (Bryson, J. S. and Flanagan, D. L., J. Hematother. Stem Cell Res, 9, 307, 2000, which is incorporated herein by reference in its entirety).

Whereas the effector or activator function of NK cells can be stimulated by cytokines including interleukin-2 (IL-2), IL-12, IL-15, IL-18, IL-21, and type I interferon (IFN-α) in combination with differential engagement of cell surface receptors, they produce immunoregulatory cytokines such as IFN-γ IL-5, IL-10, IL-13, TNF-α and granulocyte-macrophage colony-stimulating factor (GM-CSF), as well as a number of chemokines following interaction of NK receptors with their ligands (Shi F. D., Wang H. B., Li H., Hong S., Taniguchi M., Link H., Van Kaer L., Ljunggren H. G., Nat. Immunol., 1, 245, 2000). In human, the CD56^{bright} NK cell subset also produces several cytokines including IFN-γ, TNF-α TNF-β IL-10 and GM-CSF (Lian R. H., Maeda M., Lohwasser S., Delcommenne M., Nakano T., Vance R. E., Raulet D. H., Takei F., J. Immunol., 168, 4980, 2002, which is incorporated herein by reference in its entirety), but the CD56^{dim} NK cell subset do not produce these cytokines (Colucci, F., Di Santo, J. P. and Leibson, P. J., Nat. Immunol., 3, 807, 2002; Shi F. D., Wang H. B., Li H., Hong S., Taniguchi M., Link H., Van Kaer L., Ljunggren H. G., Nat. Immunol., 1, 245, 2000). Although immature NK cells can produce Th2 cytokines such as IL-5 and IL-13, the ability to produce Th2 cytokines is lost upon terminal development; instead, mature NK cells acquire the ability to produce IFN-γ (Van Beneden K., Stevenaert F., De Creus A., Debacker V., De Boever J., Plum J., Leclercq G., J. Immunol., 166, 4302, 2001, which is incorporated herein by reference in its entirety). NK cells secrete and respond to a number of chemokines including XCL1, CCL1, CCL3, CCL4, CCL5, CCL22, and CXCL8 (Lundwall, A., Dackowski, W., Cohen, E., Shaffer, M., Mahr, A., Dahlback, B., Stenclrclr, J. and Wydro, R., Proc. Natl. Acad. Sci., 83, 6716, 1986, which is incorporated herein by reference in its entirety), which are regulated, in part, by IL-15 (Crosier, K. E. and Crosier, P. S., Pathology, 29, 131, 1997; Nakano, T., Kawamoto, K., Kishino, J., Nomura, K., Higashino, K. and Arita, H., J. Biochem., 323, 387, 1997; Fridell, Y. W., Villa, J., Jr, Attar, E. C. and Liu, E. T., J. Biol. Chem., 273, 7123, 1997, each of which is incorporated herein by reference in its entirety), or IL-2 (Goruppi, S., Ruaro, E. and Schneider, C., Oncogene, 12, 471, 1996, which is incorporated herein by reference in its entirety). These chemokines play an important role in the ability of NK cells to target infected and neoplastic cells in secondary lymphoid tissues, where their production of IFN-γ can serve to directly regulate T cell responses (Lundwall, A., Dackowski, W., Cohen, E., Shaffer, M., Mahr, A., Dahlback, B., Stenclrclr, J. and Wydro, R., Proc. Natl. Acad. Sci., 83, 6716, 1986). While resting CD56^{dim}/CD16⁺ NK cell subsets express CXCR1, CXCR2, CXCR3, and CXCR4, CD56^{bight}/CD16³¹ NK cells express high levels of CCR5 and CCR7. Cytolytic activity of NK cells is stimulated by CCL2, CCL3, CCL4, CCL5, CCL10, and CXC3L1.

There are two different mechanisms known for NK cells to destroy cancer cells. According to the first mechanism, a cellular receptor is utilized. NK cells express three types of tumor necrosis factor protein on cell surface; i.e., FAS ligand (FASL), tumor necrosis factor and TRAIL, all of which are known to bind to their receptors on cancer cells to induce apoptosis of the cancer cells (Ashkenazi, A., Nature Rev. Cancer., 2, 420, 2002, which is incorporated herein by reference in its entirety). The other mechanism for NK cells to destroy cancer cells is via cytoplasmic granular materials such as perforin or granzyme. These granular materials can make a hole in cellular membrane of cancer cells and consequently destroy the cells by lysis (Trapani, J. A., Davis, J., Sutton, V. R. and Smyth, M. J., Curr. Opin. Immunol., 12, 323, 2000, which is incorporated herein by reference in its entirety).

Although it has been demonstrated that NK cells can be derived from pluripotent hematopoietic stem cells (HSCs) (Lian R. H., Maeda M., Lohwasser S., Delcommenne M., Nakano T., Vance R. E., Raulet D. H., Takei F., J. Immunol., 168, 4980, 2002), the ontogeny of NK cells has not been fully understood yet. HSCs (Lin⁻CD34⁺ in human, Lin⁻c-kit⁺Sca2⁺ in mouse) can be derived from fetal thymus, fetal liver, umbilical cord blood, and have a potential to develop into the common T/NK bipotent progenitors (Lian R. H., Kumar V., Semin. Immunol., 14, 453, 2002; Douagi I., Colucci F., Di Santo J P., Cumano A., Blood, 99, 473, 2002, each of which is incorporated herein by reference in its entirety), which are committed to pNK upon in vitro culture in combination with IL-7, stem cell factor (SCF), and flt3L (Williams N. S., Klem J., Puzanov I. J., Sivakumar P. V., Bennett M., Kumar V., J. Immunol, 163, 2648, 1999, which is incorporated herein by reference in its entirety). Here, differentiation means a phenomenon that a relatively simple system is divided into at least two qualitatively different partial systems. In other words, structure or function becomes specialized. Precursor NK cells, which are intermediate stage cells to mNK cells, are widely distributed among bone marrow, fetal thymus, blood, spleen, and liver, etc. However, as they are incapable of producing interferon-γ, they do not possess cytolytic activity. As described herein, the markers used for identification of a hematopoietic cell are c-kit⁺Lin⁻ (mouse), and CD34⁺ (human).

When pNK cells (CD56⁻CD122⁺CD34⁺ in human, CD122⁺NK1.1⁻DX5⁻ in mouse) are subsequently cultured in the presence of IL-15, the cells are able to develop into immature NK cells (CD122⁺CD161⁻CD56⁻KIR⁻ in human, CD122⁺CD2⁺NK1.1⁺DX5⁺Ly49⁻ in mouse). Prolonged culture of pNK cells with IL-15 alone can generate the pseudomature lytic NK cells (CD122⁺CD2⁺NK1.1⁺DX5⁺CD94/NKG2⁺Ly49⁻), which have partial cytolytic activities. Stromal cells provide both various cytokines and substances that can directly contact via cell surface receptors to developing NK cells, indicating that they are essential for the generation of lytic LyS49⁺ mNK cells as well as the maturation of NK cells (Iizuka K., Chaplin D. D., Wang Y., Wu Q., Pegg L. E., Yokoyama W. M., Fu Y. X., Proc. Natl. Acad. Sci., 96, 6336, 1999; Briard D., Brouty-Boye D., Azzarone B., Jasmin C., J. Immunol., 168, 4326, 2002, each of which is incorporated herein by reference in its entirety). Thus, high frequency of activated lytic Ly49⁺ mNK cells (CD56⁺KIR⁺CD3⁻ in human, CD122⁺CD2⁺NK1.1⁺DX5⁺CD94/NKG2⁺Ly49⁺CD3⁻ in mouse) arises from the co-culture of pNK cells with stromal cells in the presence of IL-15 (Williams N. S., Klem J., Puzanov I. J., Sivakumar P. V., Bennett M., Kumar V., J. Immunol., 163, 2648, 1999). During differential stages of NK cells during NK cell development, CD94, NKG2A, NKG2C and Ly49B were expressed at the early stages of development, and Ly49G, Ly49C, Ly49I in order, and finally, Ly49A, D, E and F (Williams N. S., Kubota A., Bennett M., Kumar V., Takei F., Eur. J. Immunol., 30, 2074, 2000, which is incorporated herein by reference in its entirety). As described herein, the marker used for identification of precursor NK cells is CD122⁺NK1.1⁻ (mouse).

pNK cells express transcription factors such as PU.1, GATA3, Id2, and Ets-1. Deficiency of ikaros (Boggs S. S., Trevisan M., Patrene K., Geogopoulos K., Nat. Immunol., 16, 137, 1998, which is incorporated herein by reference in its entirety), PU.1 (Colucci F., Samson S. I., DeKoter R. P., Lantz O., Singh H., Di Santo J. P., Blood, 97, 2625, 2001, which is incorporated herein by reference in its entirety), and Id2 (Ikawa T., Fujimoto S., Kawamoto H., Katsura Y., Yokota Y., Proc. Natl. Acad. Sci, 98, 5164, 2001, which is incorporated herein by reference in its entirety) causes reduced numbers of pNK cells in mice. In hematopoietic cells, transcription factors such as myeloblastosis (myb) oncogene, c-myc, and Oct 2b are expressed and they play an important role in the regulation of development and proliferation of pNK cells (Bar-Ner M., Messing L. T., Segal S., Immunobiology, 185, 150, 1992; Melotti P., Calabretta B., Blood, 87, 2221, 1996, which is incorporated herein by reference in its entirety). In pNK cells, immune regulators such as Fc receptor, TNF receptor, IL-7 receptor, chemokine receptor, and CD36 seem to have critical roles in this stage.

In mNK cells, signaling molecules such as regulator of G protein signaling (RGS), lymphocyte-specific protein tyrosine kinase, and Fyn proto-oncogene are known to be involved with NK cell maturation. (Ikawa T., Fujimoto S., Kawamoto H., Katsura Y., Yokota Y., Proc. Natl. Acad. Sci., 98, 5164, 2001; Ogasawara K., Hida S., Azimi N., Tagaya Y., Sato T., Yokochi-Fukuda T., Waldmann T. A., Taniguchi T., Taki S., Nature, 391, 700, 1998, which is incorporated herein by reference in its entirety). mNK cells refer to the cells capable of recognizing cancer cells and destroying them directly. It is known that the treatment of mNK cells with IL-2 induces the proliferation and activation of mNK cells. Receptor tyrosine kinases (RTK) constitute a large class of transmembrane proteins that relay extracellular stimuli into intracellular signals for cell proliferation, development, survival and migration (Ullrich, A., Schlessinger, J., Cell, 61, 203, 1990; Fantl, W. J., Johnson, D. E., Williams, L. T., Biochem., 62, 453, 1993; Heldin, C., Cell, 80, 213, 1995, each of which is incorporated herein by reference in its entirety). All RTK have a highly conserved cytoplasmic kinase domain that is activated upon growth factor binding to receptor monomers. The markers for mNK cells can be defined as CD122⁺NK1⁺ (mouse) and CD34⁺ (human), respectively. Other markers known in the art for NK cells that are completely matured among mNK cells can be used, such as, for example, Ly49A⁺, Ly49C⁺, Ly49D⁺, Ly49E⁺, Ly49F⁺, Ly49G⁺, Ly49H⁺, Ly49I⁺, NKG2A/C/E⁺ for mouse, and CD56⁺, NKG2A⁺, CD161⁺, NKP46⁺, NKP30⁺, NKP44⁺, and NKG20⁺ for human.

RTK activation leads to their auto-phosphorylation, and the tyrosine phosphorylation of multiple downstream intracellular signaling molecules, which are then able to initiate a variety of signal transduction cascades. The Axl receptor tyrosine kinase (named also ARK, UFO, or TYRO7) is the first discovered member of a subfamily of RTKs that share a unique structure, with extracellular regions composed of two immunoglobulin-related domains linked to two fibronectin type-III repeats, and cytoplasmic regions that contain an intrinsic tyrosine kinase domain (O'Bryan, J. P., Frye, R. A., Cogswell, P. C., Neubauer, A., Kitch, B., Prokop, C., Espinosa, R. III, Lebeau, M. M., Earp, H. S., Liu, E. T., Mol. Cell. Biol., 11, 5016, 1991, which is incorporated herein by reference in its entirety). Axl is expressed in breast, skeletal muscle, heart, hematopoietic tissue, testis, ovarian follicles and uterine endometrium (Faust, M., Ebensperger C., Schulz, A. S., Schleithoff, L., Hameister, H., Bartram, C. R. and Janssen, J. W., Oncogene, 7, 1287, 1992; Graham, D. K., Bowman G. W., Dawson, T. L., Stanford W. L., Earp, H. S., and Snodgrass, H. R., Oncogene, 10, 2349, 1995; Neubauer, A., Fiebeler, A., Graham, D. K., O'Bryan, J. P., Schmidt, C. A., Barckow, P., Serke, S., Siegert, W., Snodgrass, H. R., Huhn, D., Blood, 84, 1931, 1994; Berclaz, G., Altermatt, H. J., Rohrbach, V., Kieffer, I., Dreffer, E. and Andres, A. C., Ann. Oncol., 12, 819, 2001; Wimmel, A., Glitz, D., Kraus, A., Roeder, J. and Schuermann, M., Eur. J. Cancer., 37, 2264, 2001; Sun, W. S., Misao, R., Iwagaki, S., Fujimoto, J. and Tamaya, T., Mol. Hum. Reprod., 8, 552, 2002, each of which is incorporated herein by reference in its entirety). Axl is typified by the cell adhesion molecule-related extracellular ligand-binding domain, composed of two immunoglobulin-like motifs and two fibronectin type III motifs. Recent studies suggest a role of Axl in developmental processes of the hematopoietic and nervous systems, and in tumorigenesis (Crosier, K. E. and Crosier, P. S., Pathology, 29, 131, 1997).

Gas6, the protein product of growth arrest-specific gene 6 (Gas6), is a member of the vitamin K-dependent protein family that was identified as a ligand for the Axl/Sky family of receptor tyrosine kinases including Axl, Sky (Rse, Brt, Tif, Dtk, Etk-2 and Tyro3) and Mer (c-Eyk, Nyk and Tyro12) (Godowski, P. J., Mark, M. R., Chen, J., Sadick, M. D., Raab, H. and Hammonds R. G., Cell, 82, 355, 1995; Varnum, B. C., Young, C., Elliott, G., Garcia, A., Bartley, T. D., Fridell, Y. W., Hunt, R. W., Trail, G., Clogston, C., Toso, R. J., Nature, 373, 623, 1995; Chen, J., Carey, K. and Godowski, P. J., Oncogene, 14, 2033, 1997, each of which is incorporated herein by reference in its entirety). It is known that Gas6 has approximately 46% amino acid identity to protein S, a serum protein that negatively regulates blood coagulation (Manconrftti, G., Brancolini, C., Avanzi, G. and Schneider, C., Mol. Cell. Biol., 13, 4976, 1993, which is incorporated herein by reference in its entirety). Gas6 is expressed in the intestine, testicular somatic cells, pulmonary endothelium and uterine endometrium, and in uterineendometrial cancers (Prieto, A. L., Weber, J. L., Tracy, S., Heeb, M. J. and Lai, C., Brain Res., 816, 646, 1999; Chan, M. C. W., Mather, J. P., Mccray, G. and Lee, W. M., J. Androl., 21, 291, 2000; Wimmel, A., Glitz, D., Kraus, A., Roeder, J. and Schuermann, M., Eur. J. Cancer., 37, 2264, 2001, each of which is incorporated herein by reference in its entirety).

The production of Gas6 with full biological activity can include a vitamin K-dependent post-translational modification in which the newly synthesized peptide is extensively γ-carboxylated in the endoplasmic reticulum (Manconrftti, G., Brancolini, C., Avanzi, G. and Schneider, C., Mol. Cell. Biol., 13, 4976, 1993; Lundwall, A., Dackowski, W., Cohen, E., Shaffer, M., Mahr, A., Dahlback, B., Stenclrclr, J. and Wydro, R., Proc. Natl. Acad. Sci., 83, 6716, 1986; Chen, J., Carey, K. and Godowski, P. J., Oncogene, 14, 2033, 1997). Gas6 acts as a growth-potentiating factor for thrombin-induced proliferation of vascular smooth muscle cells (Nakano, T., Kawamoto, K., Kishino, J., Nomura, K., Higashino, K. and Arita, H., J. Biochem., 323, 387, 1997). Additionally, Gas6 has been shown to be a novel chemoattractant that induces Axl-mediated migration of vascular smooth muscle cells (Davis, J. E., Smyth, M. J. and Trapani, J. A., Eur. J. Immunol., 31, 39-47, 2001, which is incorporated herein by reference in its entirety). Gas6 is also able to induce cell cycle re-entry and protect serum-starved NIH3T3 cells from apoptotic cell death (Goruppi, S., Ruaro, E. and Schneider, C., Oncogene, 12, 471, 1996). Recently, Gas6 has been shown to induce beta-catenin stabilization and T-cell factor transcriptional activation in mammary cells (Goruppi, S., Chiaruttini, C., Ruaro, M. E., Varnum, B. and Schneider, C., Mol. Cell. Biol., 21, 902, 2001, which is incorporated herein by reference in its entirety).

Protein S is a vitamin-K dependent plasma glycoprotein that serves as an important cofactor for activated protein C in the blood anticoagulation system. Protein S also acts as a mitogen on distinct cell types and is a ligand for Tyro3, a member of the Axl family of oncogenic receptor tyrosine kinases (Wimmel A. et al., Cancer 1999 Jul. 1; 86(1):43-9, which is incorporated herein by reference in its entirety). It is known that Protein S stimulates a member of Axl/Sky protein, similar to Gas 6. Protein S has a similar structure to Gas 6 (i.e., it is constituted with a Gla domain at the N-terminus, four EGF-like domains, and a G domain similar to a signal transduction molecule) and can phosphorylate Axl/Sky protein family (Evenas, P., et al., Biol. Chem. 2000 March; 381(3):199-209, which is incorporated herein by reference in its entirety).

Protein S exists in two forms in human plasma, namely as the free protein and also in complex with C4b-binding protein (Dahlback & Stenflo (1981) Proc. Natl. Acad. Sci. U.S.A. 78, 2512-2516, which is incorporated herein by reference in its entirety). Protein S can be isolated by several simple purification methods, which include for example, barium citrate adsorption, DEAE-Sephacel chromatography and chromatography on Blue Sepharose (Dahlback B., Biochem. J. 1983 Mar. 1; 209(3):837-846, which is incorporated herein by reference in its entirety). In addition, Protein S can be produced by genetic recombination method (Merel Van Wijnen, et al., Biochem. J. 330, 389-396, which is incorporated herein by reference in its entirety).

Recombinant DNA methods used herein are generally, those set forth in Sambrook et al. (Molecular Cloning: A Laboratory Manual, Cold Spring Harbor Laboratory Press, Cold Spring Harbor, N.Y. (1989), which is incorporated herein by reference in its entirety) and/or Ausubel et al., eds., (Current Protocols in Molecular Biology, Green Publishers Inc. and Wiley and Sons, N.Y. (1994), which is incorporated herein by reference in its entirety). For example, by inserting a nucleic acid sequence which encodes the amino acid sequence of Axl, Gas6 or protein S polypeptide into an appropriate vector, one skilled in the art can readily produce large quantities of the desired nucleotide sequence. The sequences can then be used to generate detection probes or amplification primers. Alternatively, a polynucleotide encoding the amino acid sequence of Axl, Gas6 or protein S polypeptide can be inserted into an expression vector. By introducing the expression vector into an appropriate host, the encoded Axl, Gas6 or protein S polypeptide can be produced in large amounts. Another method for obtaining a suitable nucleic acid sequence is the polymerase chain reaction (PCR). In this method, cDNA is prepared from poly(A)+ RNA or total RNA using the enzyme reverse transcriptase. Two primers, typically complementary to two separate regions of cDNA (oligonucleotides) encoding the amino acid sequence of Axl, Gas6 or protein S polypeptide, are then added to the cDNA along with a polymerase such as Taq polymerase, and the polymerase amplifies the cDNA region between the two primers. A nucleic acid molecule encoding the amino acid sequence of Axl, Gas6 or protein S polypeptide can be amplified/expressed in prokaryotic, yeast, insect (baculovirus systems), and/or eukaryotic host cells (Meth. Enz. vol. 185 D. V. Goeddel ed., Academic Press, San Diego Calif., 1990, which is incorporated herein by reference in its entirety). Typically, expression vectors used in any of the host cells will contain sequences for plasmid maintenance and for cloning and expression of exogenous nucleotide sequences. Such sequences include a promoter, an enhancer sequence, an origin of replication, a transcriptional termination sequence, a sequence encoding a leader sequence for secretion, a ribosome binding site, a polyadenylation sequence, a polylinker region for inserting the nucleic acid encoding the polypeptide to be expressed, and a selectable marker element, etc. Such flanking sequences are all well known to a skilled person in the pertinent art and can be easily selected by him.

Examples of suitable promoters for directing the transcription of the DNA encoding the human Axl, Gas6 or protein S in mammalian cells are the SV40 promoter (Subramani et al., Mol. Cell. Biol. 1 (1981), 854-864, which is incorporated herein by reference in its entirety), the MT-1 (metallothionein gene) promoter (Palmiter et al., Science 222 (1983), 809-814, which is incorporated herein by reference in its entirety), the CMV promoter (Boshart et al., Cell 41:521-530, 1985, which is incorporated herein by reference in its entirety) or the adenovirus 2 major late promoter (Kaufman and Sharp, Mol. Cell. Biol, 2:1304-1319, 1982, which is incorporated herein by reference in its entirety). An example of a suitable promoter for use in insect cells is the polyhedrin promoter (U.S. Pat. No. 4,745,051; Vasuvedan et al., FEBS Lett. 311, (1992) 7-11, each of which is incorporated herein by reference in its entirety), the P10 promoter (J. M. Vlak et al., J. Gen. Virology 69, 1988, pp. 765-776, which is incorporated herein by reference in its entirety), the baculovirus immediate early gene 1 promoter (U.S. Pat. No. 5,155,037; U.S. Pat. No. 5,162,222, each of which is incorporated herein by reference in its entirety), or the baculovirus 39K delayed-early gene promoter (U.S. Pat. No. 5,155,037; U.S. Pat. No. 5,162,222, each of which is incorporated herein by reference in its entirety). Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434, each of which is incorporated herein by reference in its entirety) or alcohol dehydrogenase genes (Young et al., in Genetic Engineering of Microorganisms for Chemicals (Hollaender et al, eds.), Plenum Press, New York, 1982, which is incorporated herein by reference in its entirety), or the TPI1 (U.S. Pat. No. 4,599,311, which is incorporated herein by reference in its entirety) or ADH2-4c (Russell et al., Nature 304 (1983), 652-654, which is incorporated herein by reference in its entirety) promoters. Examples of suitable promoters for use in filamentous fungus host cells are, for instance, the ADH3 promoter (McKnight et al., The EMBO J. 4 (1985), 2093-2099, which is incorporated herein by reference in its entirety) or the tpiA promoter. Examples of other useful promoters are those derived from the gene encoding A olyzae TAKA amylase, Rhizomucor miehei aspartic proteinase, A. niger neutral.alpha.-amylase, A. niger acid stable o-amylase, A. niger or A. awamori glucoamylase (gluA), Rhizomucor miehei lipase, A. oryzae alkaline protease, A. oryzae triose phosphate isomerase or A. nidulans acetamidase.

The DNA sequences can also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. Expression vectors can also contain a set of RNA splice sites located downstream from the promoter and upstream from the insertion site for the DNA sequence itself. Preferred RNA splice sites can be obtained from adenovirus and/or immunoglobulin genes. Also contained in the expression vectors is a polyadenylation signal located downstream of the insertion site. Particularly preferred polyadenylation signals include the early or late polyadenylation signal from SV40, the polyadenylation signal from the adenovirus 5 E1b region or the human growth hormone gene terminator (DeNoto et al. Nucl. Acids Res. 9:37193730, 1981, which is incorporated herein by reference in its entirety). The expression vectors can also include a noncoding viral leader sequence, such as the adenovirus 2 tripartite leader, located between the promoter and the RNA splice sites; and enhancer sequences, such as the SV40 enhancer.

To direct the human polypeptide of the present invention into the secretory pathway of the host cells, a secretory signal sequence (also known as a leader sequence, prepro sequence or pre sequence) can be provided in the recombinant vector. The secretory signal sequence is joined to the DNA sequences encoding the human polypeptide in the correct reading frame. Secretory signal sequences are commonly positioned 5′ to the DNA sequence encoding the peptide. The secretory signal sequence can be that normally associated with the protein, or it can be from a gene encoding another secreted protein. For secretion from yeast cells, the secretory signal sequence can encode any signal peptide, which ensures efficient direction of the expressed human polypeptide into the secretory pathway of the cell. The signal peptide can be a naturally occurring signal peptide, or a functional part thereof, or it can be a synthetic peptide. Suitable signal peptides can include, for example, the α-factor signal peptide (U.S. Pat. No. 4,870,008, which is incorporated herein by reference in its entirety), the signal peptide of mouse salivary amylase (O. Hagenbuchle et al., Nature 289, 1981, pp. 643-646, which is incorporated herein by reference in its entirety), a modified carboxypeptidase signal peptide (L. A. Valls et al., Cell 48, 1987, pp. 887-897, which is incorporated herein by reference in its entirety), the yeast BAR1 signal peptide (WO 87/02670, which is incorporated herein by reference in its entirety), or the yeast aspartic protease 3 (YAP3) signal peptide (M. Egel-Mitani et al., Yeast 6, 1990, pp. 127-137, which is incorporated herein by reference in its entirety). For efficient secretion in yeast, a sequence encoding a leader peptide can also be inserted downstream of the signal sequence and upstream of the DNA sequence encoding the human polypeptide. The function of the leader peptide is to allow the expressed peptide to be directed from the endoplasmic reticulum to the Golgi apparatus and further to a secretory vesicle for secretion into the culture medium (i.e. exportation of the human polypeptide across the cell wall or at least through the cellular membrane into the periplasmic space of the yeast cell). The leader peptide can be the yeast alpha-factor leader (the use of which is described in e.g. U.S. Pat. No. 4,546,082, U.S. Pat. No. 4,870,008, EP 16 201, EP 123 294, EP 123 544 and EP 163 529, each of which is incorporated herein by reference in its entirety). Alternatively, the leader peptide can be a synthetic leader peptide, which is to say a leader peptide not found in nature. Synthetic leader peptides can, for instance, be constructed as described in WO 89/02463 or WO 92/11378, each of which is incorporated herein by reference in its entirety. For use in filamentous fungi, the signal peptide can conveniently be derived from a gene encoding an Aspergillus sp. amylase or glucoamylase, a gene encoding a Rhizomucor miehei lipase or protease or a Humicola lanuginosa lipase. For use in insect cells, the signal peptide can conveniently be derived from an insect gene (WO 90/05783, which is incorporated herein by reference in its entirety), such as the lepidopteran Manduca sexta adipokinetic hormone precursor signal peptide (U.S. Pat. No. 5,023,328, which is incorporated herein by reference in its entirety).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet 1 (1982), 327-341; Loyter et al., Proc. Natl. Acad. Sci. USA 79 (1982), 422-426; Wigler et al., Cell 14 (1978), 725-732; Corsaro and Pearson, Somatic Cell Genetics 7 (1981), 603; Graham and van der Eb, Virology 52 (1973), 456-467; and Neumann et al., EMBO J. 1 (1982), 841-845, each of which is incorporated herein by reference in its entirety. Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker can be an amplifiable selectable marker. A preferred amplifiable selectable marker is a dihydrofolate reductase (DHFR) sequence. Selectable markers can be introduced into the cell on a separate plasmid at the same time as the gene of interest, or they can be introduced on the same plasmid. If, on the same plasmid, the selectable marker and the gene of interest can be under the control of different promoters or the same promoter, the latter arrangement producing a dicistronic message. Constructs of this type are known in the art (for example, U.S. Pat. No. 4,713,339, which is incorporated herein by reference in its entirety). It can also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components for the growth of cells and the expression of the human polypeptide of interest. Media generally include a carbon source, a nitrogen source, essential amino acids, essential sugars, vitamins, salts, phospholipids, protein and growth factors. For production of γ-carboxylated proteins, the medium will contain vitamin K, preferably at a concentration of from about 0.1 μg/ml to about 5 μg/ml. Drug selection is then applied to select for the growth of cells that are expressing the selectable marker in a stable fashion. For cells that have been transfected with an amplifiable selectable marker the drug concentration can be increased to select for an increased copy number of the cloned sequences, thereby increasing expression levels. Clones of stably transfected cells are then screened for expression of the human polypeptide of interest.

The host cell into which the DNA sequences encoding the human polypeptide of interest is introduced can be any cell, which is capable of producing the posttranslational modified human polypeptide and includes yeast, fungi and higher eukaryotic cells. Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977, which is incorporated herein by reference in its entirety) cell lines. A preferred BHK cell line is the tk⁻ts13 BHK cell line (Waechter and Baserga, Proc. Natl. Acad. Sci. USA 79:1106-1110, 1982, which is incorporated herein by reference in its entirety). In addition, a number of other cell lines can be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980, which is incorporated herein by reference in its entirety). Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides therefrom are described, e.g. in U.S. Pat. No. 4,599,311, U.S. Pat. Nos. 4,870,008, 5,037,743, and U.S. Pat. No. 4,845,075, each of which is incorporated herein by reference in its entirety. Transformed cells are selected by a phenotype determined by a selectable marker, commonly drug resistance or the ability to grow in the absence of a particular nutrient, e.g. leucine. A preferred vector for use in yeast is the POT 1 vector disclosed in U.S. Pat. No. 4,931,373, which is incorporated herein by reference in its entirety. Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 43 8, each of which is incorporated herein by reference in its entirety. The transformation of F. oxysporum can, for instance, be carried out as described by Malardier et al., 1989, Gene 78: 147-156, which is incorporated herein by reference in its entirety. The transformation of Trichoderma spp. can be performed for instance as described in EP 244 234, which is incorporated herein by reference in its entirety. When a filamentous fungus is used as the host cell, it can be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome can be performed according to conventional methods, e.g. by homologous or heterologous recombination. Transformation of insect cells and production of heterologous polypeptides therein can be performed as described in U.S. Pat. No. 4,745,051; U.S. Pat. No. 4,879,236; U.S. Pat. Nos. 5,155,037; 5,162,222; EP 397,485, each of which is incorporated herein by reference in its entirety. The insect cell line used as the host can suitably be a Lepidoptera cell line, such as Spodoptera frugiperda cells or Trichoplusia ni cells (U.S. Pat. No. 5,077,214, which is incorporated herein by reference in its entirety). Culture conditions can suitably be as described in, for instance, WO 89/01029 or WO 89/01028, which is incorporated herein by reference in its entirety.

The transformed or transfected host cell described above is then cultured in a suitable nutrient medium under conditions permitting expression of the human polypeptide after which all or part of the resulting peptide can be recovered from the culture. The medium used to culture the cells can be any conventional medium suitable for growing the host cells, such as minimal or complex media containing appropriate supplements. Suitable media are available from commercial suppliers or can be prepared according to published recipes (e.g. in catalogues of the American Type Culture Collection). The proteins produced by the cells can then be recovered from the culture medium by, conventional procedures including separating the host cells from the medium by centrifugation or filtration, precipitating the aqueous protein components of the supernatant or filtrate by means of a salt, e.g. ammonium sulphate, purification by a variety of chromatographic procedures, e.g. ion exchange chromatography, gel filtration chromatography, affinity chromatography, or the like, dependent on the type of polypeptide in question.

A transfectant is defined as a transfected host cell that has been transformed or transfected as described above. It can also refer to a host cell that has been transformed by infection with viruses, such as, for example, retroviruses, lentiviruses, adenoviruses, adeno-associated viruses, and any virus that is well known in the art for use in transformation of host cells. A transfectant can also describe a host cell that has been transformed by infection with viral vectors that are derived from any of the viruses described above.

Axl antibody used according to the present invention can be either polyclonal or monoclonal. Axl antibody can be purchased from Santa Cruz Biotech. The antibody used in the present invention can be prepared based on a publicly known method.

The term antibody in its various grammatical forms is used herein to refer to immunoglobulin molecules and immunologically active portions of immunoglobulin molecules of the compositions of this invention, i.e., molecules that contain an antibody combining site or paratope. Exemplary antibody molecules are intact immunoglobulin molecules, substantially intact immunoglobulin molecules and portions of an immunoglobulin molecule, including those portions known in the art as Fab, Fab′, F(ab′)₂ and Fv. The term “antibody” as used herein is also intended to include human, single chain and humanized antibodies, as well as binding fragments of such antibodies or modified versions of such antibodies, such as multispecific, bispecific and chimeric molecules having at least one antigen binding determinant derived from an antibody molecule. A chimeric antibody is one in which the variable regions are from one species of animal and the constant regions are from another species of animal. For example, a chimeric antibody can be an antibody having variable regions which derive from a mouse monoclonal antibody and constant regions which are human.

Depending on the amino acid sequences of the constant domain of their heavy chains, immunoglobulins can be assigned to different classes. There are at least five (5) major classes of immunoglobulins: IgA, IgD, IgE, IgG and IgM, and several of these can be further divided into subclasses (isotypes), e.g. IgG-1, IgG-2, IgG-3 and IgG-4; IgA-1 and IgA-2. The heavy chains constant domains that correspond to the different classes of immunoglobulins are called alpha (α), delta (δ), epsilon (ε), gamma (γ) and mu (μ), respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

Polyclonal antibodies are a mixture of antibody molecules recognising a specific given antigen, hence polyclonal antibodies can recognise different epitopes within said antigen. Polyclonal antibodies are obtained by subcutaneous or peritoneal injection of an antigen and an adjuvant to an animal. It can be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester, N-hydroxysuccinimide, glutaraldehyde, succinic anhydride or SOCl₂. Animals are immunized against the antigen, immunogenic conjugates, or derivatives by combining, e.g., approximately 100 μg or approximately 5 μg of the protein or conjugate (for rabbits or mice, respectively) with about 3 volumes of Freund's complete adjuvant and injecting the solution intradermally at multiple sites. About one month later the animals are boosted with from about ⅕ to about 1/10 the original amount of peptide or conjugate in Freund's complete adjuvant by subcutaneous injection at multiple sites. From about seven to about fourteen days later the animals are bled and the serum is assayed for antibody titer. Animals are boosted until the titer plateaus. Preferably, the animal is boosted with the conjugate of the same antigen, but conjugated to a different protein and/or through a different cross-linking reagent can be also used. Conjugates also can be made in recombinant cell culture as a fusion protein. Also, aggregating agents such as alum are suitable used to enhance the immune response.

The phrase monoclonal antibody in its various grammatical forms refers to a population of antibody molecules that contains only one species of antibody combining site capable of immunoreacting with a particular antigen. A monoclonal antibody thus typically displays a single binding affinity for any antigen with which it immunoreacts. A monoclonal antibody can contain an antibody molecule having a plurality of antibody combining sites, each immunospecific for a different antigen, e.g., a bispecific monoclonal antibody. Monoclonal antibodies are obtained from a population of substantially homogeneous antibodies, i.e., the individual antibodies comprising the population are identical except for possible naturally occurring mutations that can be present in minor amounts. Thus, the “monoclonal” indicates the character of the antibody as not being a mixture of discrete antibodies, but being able to specifically recognize a single antigen.

For example, the monoclonal antibodies can be prepared using the hybridoma method first described by Kohler et al., Nature, 256:495 (1975) (which is incorporated herein by reference in its entirety), or can be prepared by recombinant DNA methods (U.S. Pat. No. 4,816,567, which is incorporated herein by reference in its entirety). In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59 103 (Academic Press, 1986), which is incorporated herein by reference in its entirety). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium). These substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51 63 (Marcel Dekker, Inc., New York, 1987), each of which is incorporated herein by reference in its entirety). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). The binding affinity of the monoclonal antibody can, for example, be determined by the Scatchard analysis of Munson et al., Anal. Biochem., 107:220 (1980) (which is incorporated herein by reference in its entirety). After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59 103 (Academic Press, 1986)). Suitable culture media for this purpose include, for example, D-MEM or RPMI-1640 medium. In addition, the hybridoma cells can be grown in vivo as ascites tumors in an animal. The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

Hematopoietic stem cells (HSCs), with capacity to renew themselves, are the source of committed progenitor cells throughout the lifetime of an individual. A general feature of primitive as well as more committed hematopoietic cells is the expression of the CD34 antigen, which can be detected by FACS (fluorescence-activated cell sorter) analysis using anti-CD34 monoclonal antibodies. HSC is derived from bone marrow, peripheral blood, umbilical cord blood, etc. Umbilical cord blood (UCB) is known to be a rich source of hematopoietic stem cells (SCs). UCB, obtained from the placenta directly after delivery, is enriched in SCs and has a higher proliferative capacity than cells obtained from bone marrow and peripheral blood. The introduction of hematopoietic growth factors such as G-CSF has greatly facilitated the mobilization of CD34(+) cells (Beyer J et al., Hematopoietic rescue after high-dose chemotherapy using autologous peripheral-blood progenitor cells or bone marrow: a randomized comparison. J Clin Oncol 1995; 13:1328-1335; and Smith T J et al., Economic analysis of a randomized clinical trial to compare filgrastim-mobilized peripheral-blood progenitor-cell transplantation and autologous bone marrow transplantation in patients with Hodgkin's and non-Hodgkin's lymphoma. J Clin Oncol 1997; 15:5-10, each of which is incorporated herein by reference in its entirety). G-CSF is administered in dosage of about 300-960 μg/d until the end of the collection period. Strategies employed to isolate or purify such cells are numerous, and these include sorting of cells according to specific cell surface markers using either fluorescence (Preffer F I, et al., Lineage-negative side-population(SP) cells with restricted hematopoietic capacity circulate in normal human adult blood: immunophenotypic and functional characterization. Stem Cells 2002; 20:417-427, which is incorporated herein by reference in its entirety) or immunomagnetic technology (Przyborski S A. Isolation of human embryonal carcinoma stem cells by immunomagnetic sorting. Stem Cells 2001; 19:500-504, which is incorporated herein by reference in its entirety), exploiting the differential plating efficiencies of stem cells on culture plastic (Friedenstein A J, et al., Fibroblast precursors in normal and irradiated mouse hematopoietic organs. Exp Hematol 1976; 4:267-274, which is incorporated herein by reference in its entirety), and column-separation techniques (Huss R. Isolation of primary and immortalized CD34 hematopoietic and Mesenchymal stem cells from various sources. Stem Cells 2000; 18:19, which is incorporated herein by reference in its entirety).

It is well known to use IL-2 to activate mNK cells. Two different methods are suggested for the treatment with IL-2. First, IL-2 is directly administered to a patient so that NK cells are proliferated and activated by IL-2 in the patient's own body. Second, blood is drawn from the patient and mNK cells are isolated from the blood and activated by IL-2, and then the resulting activated mNK cells are introduced back to the patient to be treated. Both of these methods aim to destroy cancer cells by NK cells that have been activated by IL-2. However, these methods involve using high dose of IL-2 (about 150 ng/ml), which can bring an adverse effect. Specifically, high toxicity, fever, pulmonary edema, and a shock can occur, because IL-2 causes T-lymphocyte to stimulate tumor necrosis factors or other cytokines such as IL-γ so that the cytokines start interacting with vascular endothelial cells and other cells. In order to solve such problems, studies are being carried out for using low dose of IL-2 but a satisfactory result is yet to be found (M. J. Smyth, Y. Hayakawa, K. Takeda, H. Yagita, Nat Rev Cancer. 2, 850, 2002; M. A. Caligiuri, et al., J. Exp. Med. 171, 1509, 1990, each of which is incorporated herein by reference in its entirety).

According to an embodiment of the present invention, it is found that a low dose of about 8 to about 15 ng/ml of IL-2 is enough for full stimulation and activation of differentiated mNK cells. Preferred dose of IL-2 is about 10 ng/ml. Such low dose of IL-2 appears not to induce any toxicity from activated mNK cells.

Several methods are used for the preservation of cells, of which the best known is cryopreservation. In addition, HypoThermosol (BioLife Solutions Inc) family or dimethyl sulfoxide (DMSO) solutions can be used.

According to an additional aspect of the present invention, NK cells can be preserved at ultra low temperature before and after the administration to a patient. A typical method for the preservation at ultra low temperature in small scale is described in U.S. Pat. No. 6,0168,991. For small-scale preservation at ultra low temperature, cells can be re-suspended at a concentration of about 200×10⁶/ml in 5% human albumin serum (HAS) which is previously cooled. Next, an equivalent amount of 20% DMSO is added into said HAS solution. Then, aliquots of the mixture are taken into 1 ml vial and frozen overnight inside the ultra low temperature chamber (Nalgene™) at about −80° C. In case of ultra low temperature preservation in large scale, cells can be re-suspended at about 600×10⁶/ml in AIM V. Subsequently, the same amount of 20% AIM V is added gradually into the re-suspension. The resulting mixture is frozen inside freezer vessel (Cryocyte, Baxter) using speed-controlled freezing system (Forma™). Effective amount of activated mNK cells for cytotoxicity can be varied depending on use of the cells inside test tube or living human body, as well as the amount and type of cells which are ultimate target of the activated mNK cells.

As used herein, effective amount for cytotoxicity is defined as amount of mNK cell cells that is able to destroy cancer cells (or, is able to cause a pharmaceutical action). Since such effective amount can vary depending on health and severeness of a patient, it should be determined by a physician after considering all of such variables. Generally, about 10⁶ to about 10¹² cells, preferably from about 10⁸ to about 10¹¹ cells, more preferably from about 10⁹ to about 10¹⁰ cells are administered per each administration to an adult cancer patient. mNK cells proliferated by the method according to the present invention can be administered to a patient subcutaneously, intramuscular, intravenous, and epidurally, together with a pharmaceutically acceptable vehicle (for example, saline solution) for the treatment. Use of various bio materials (cell carrier) can increase the efficiency of mNK cells to be delivered to a target site and to destroy cancer cells. Cell carrier includes methylcellulose of polysaccharides (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P(NIPAM-co-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D,L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety), etc. and they are used as a cell carrier for cellular treatment in the field of tissue engineering.

The following examples and drawings are provided for illustration of the invention. They should not be considered as limiting the scope of the invention, but merely as being representative thereof.

### EXAMPLE 1

### Discovery of Specific Genes to be Expressed During the Development of Murine Natural Killer Cells

(A) Isolation of Hematopoietic Stem Cells from Murine Bone Marrow and Differentiation into Natural Killer Cells

Whole bones of 8-12 year old mouse (C57BL/6 purchased from Korea Research Bioscience and Biotechnology) were procured under the germ free condition according to animal care and handling guidelines. Bone marrow cells were isolated from those bones and treated with cell lysis buffer (0.2% NaCl, 1.6% NaCl) to remove red blood cells. The remaining cells were reacted with 2.4G2 supernatant over an ice bath and washed with phosphate-buffered solution containing 2 mM EDTA (buffer A). The cells were suspended in buffer A at the concentration of 1×10⁸/500 μL. The resulting suspension was reacted with a biotin-conjugated antibody cocktail (Mac-1, Gr-1, B220, NK 1.1, CD2, TER-119, Pharmingen) at the temperature of 4° C. for 10 minutes. The cells were washed with buffer A. 1×10⁸ cells were reacted with 900 μL of buffer A and 100 μL of streptavidin-microbeads (Miltenyi Biotec) at the temperature of 4° C. for 15 minutes. After washing, the cells were suspended in MACS (magnetic bead-activated cell sorting) buffer solution (phosphate-buffered solution containing 2 mM EDTA and 0.5% BSA) and filtered over nylon mesh (70 μm). Magnetic column (CS column, Miltenyi Biotech) was fixed onto super MACS and magnetic bead-labeled cells were passed. The column was sufficiently washed with MACS buffer and the eluted solution was centrifuged to afford lineage negative (Lin⁻) cells. FITC-conjugated anti-c-kit (Pharmingen) was added to Lin⁻ cells (1×10⁷) and the reaction was incubated at the temperature of 4° C. for 10 minutes. After washing, Lin⁻ cells were again reacted with anti-FITC microbeads (Miltenyi Biotec) at the temperature of 4° C. for 15 minutes and suspended in 500 μL of MACS buffer. The cell suspension was passed through an MS column housed within a magnetic field. The column was sufficiently washed with 1 mL of MACS buffer to elute c-kit+ cells which were used as hematopoietic cells. The isolated hematopoietic cells were cultured for 6 days in 24-well plate at the concentration of 1×10⁶/mL while half of RPMI (Gibco) medium containing SCF (30 ng/mL, Biosource), IL-7 (0.5 ng/mL, PeproTech), Flt3L (50 ng/mL, PeproTech), indometacine (2 μg/mL, Sigma), and gentamicin ((2 μg/mL, Sigma) was replaced with fresh medium every three days. After 6-day culture with said cytokines, the cultured cells were reacted with CD122-FITC antibody and multisort microbeads and MACS was carried out to isolate CD122⁺ precursor natural killer (pNK) cells. The pNK cells were then differentiated into mature natural killer cells for 6 days by two different methods using RPMI containing IL-15 (20 ng/mL), indometacine (2 μg/mL), and gentamicin (2 μg/mL). One method for the differentiation into mature natural killer cells (mNK-2) was performed by co-culturing with stromal cells (ATCC). The other method was conducted without co-culture to afford less mature natural killer cells (mNK-1). These NK cells were collected to analyze the expression of cell surface markers (FIG. 1).

(B) Purity of Stage-Separated Cells During the Differentiation from Murine Hematopoietic Stem Cells to Mature Natural Killer Cells

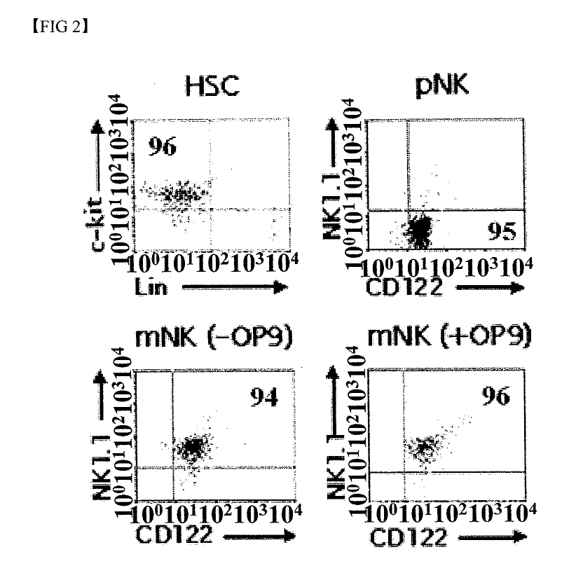

As described in the above Example 1 (A), whole 2×10⁸ bone marrow cells were obtained from eight 8-12 year old mice (C57BL/6) and Lin−, c-kit⁺ hematopoietic stem cells were separated. CD122⁺ pNK cells were obtained from hematopoietic stem cells, and were co-cultured with or without stromal cells (i.e., +OP9 and −OP9, respectively) in the presence of IL-15 to produce mNK cells. The purity of stage-separated cells was determined as follows. For immunostaining of the stage-separated cells during differentiation from hematopoietic stem cells to mNK cells with antibodies directed against various cell-associated surface molecules, 1×10⁶ cells were counted and washed once with staining buffer solution (phosphate-buffered solution containing 20 mM HEPES, 3% fetal bovine serum, 0.1% NaN₃, pH 7.4). The cells were then incubated with various NK cell differentiation-stage markers (cell markers) conjugated with FITC (fluoresceinated isothiocyanate) or PE (phycoerythrin), i.e., antibodies such as c-kit (Pharmingen), lineage (Pharmingen), NK1.1 (Pharmingen), CD122 (Pharmingen), at the temperature of 0° C. for 30 minutes. After washing twice with staining buffer solution, cells were analyzed using FACS (BD/Aria). For FACS analysis, c-kit and lineage (Pharmingen) antibodies were used for hematopoietic stem cells (c-kit⁺Lin⁻), while CD122 (Pharmaingen) and NK1.1 (Pharmingen) antibodies were used for pNK cell (CD122⁺NK1.1⁻) and mNK cells (CD122⁺NK1.1⁺). The purity of hematopoietic stem cells, pNK cells, mNK cells (−OP9) and mNK cells (+OP9) was confirmed as 96%, 95%, 94%, and 96%, respectively (FIG. 2).

(C) Identification of Genes Specifically Expressed in Mouse Natural Killer Cells

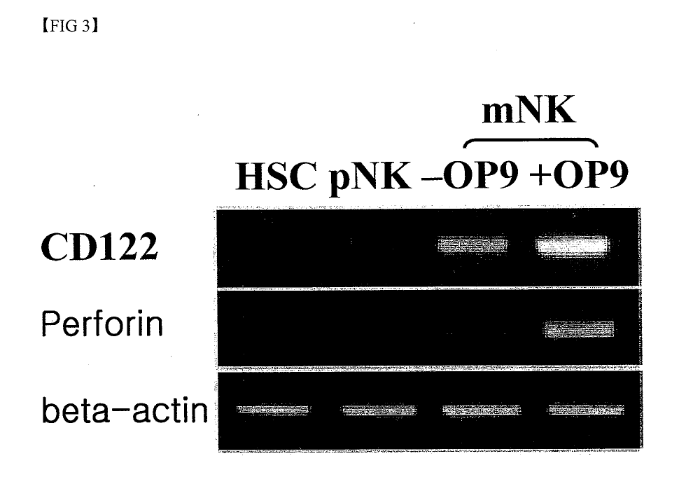

The expression of CD122 and perforin specific in natural killer cells obtained by the above step was confirmed by reverse transcriptase polymerase chain reaction (RT-PCR). The RT-PCR was performed as follows. 2×10⁶ LK1 cells were washed once with phosphate-buffered solution. After 500 μL of RNA extraction solution (RNAzol B, TEL-TEST) was added to each specimen, cells were lysed by gentle pippetting. After the addition of 50 μL of chloroform, the suspension was mixed well and stored on ice for 5 minutes. It was centrifuged at 12,000×g, 4° C. for 15 minutes. The supernatant was removed and the same volume of isopropyl alcohol was added. The resulting mixture was stored on ice for 20 min. Then, it was centrifuged again at 12,000×g, 4° C. for 20 minutes. The resulting pellet was washed with 80% ethanol. After drying the pellet, the pellet was dissolved in 20 μL of water containing 0.1% diethyl pyrocarbonate. From the RNA obtained thereby, single-stranded cDNAs were synthesized with MMLV reverse transcriptase (Roche). Using gene-specific primers (CD122: 5′-gtcgacgctcctctcagctgtgatggctaccata-3′ (SEQ ID NO: 1) and 5′-ggatcccagaagacgtctacgggcctcaaattccaa-3′ (SEQ ID NO: 2); perforin: 5′-gtcacgtcgaagtacttggtg-3′ (SEQ ID NO: 3) and 5′-aaccagccacatagcacacat-3′ (SEQ ID NO: 4); β-actin: 5′-gtggggcgccccaggcacca-3′ (SEQ ID NO: 5) and 5′-ctccttaatgtcacgcacgatttc-3′ (SEQ ID NO: 6)), RT-PCR was conducted as follows. A total 20 μL of a mixture consisting of 5 μL of 5× reaction buffer solution (Roche), 2 μL of dNTP (dATP, dCTP, dGTP, dTTP, each 5 mM, Roche), 1 μL (20 pmol) of 3 primer, 1 μL of distilled water, 10 μL of whole cellular RNA, and 1 μL (200 U) of reverse transcriptase was incubated at the temperature of 42° C. for 30 minutes and, then, quenched at the temperature of 90° C. for 5 minutes. For the PCR, the resulting solution (20 μL) was mixed with 8 μL of 10×PCR buffer solution (Roche), 1 μL (20 pmol) of 5 primer, 1 μL (20 pmol) of 3 primer, 69 μL of distilled water, and 1 μL (2.5 U) of Taq polymerase (Takara). A total 100 μL of the mixture was incubated at the temperature of 95° C. for 5 minutes to inhibit any enzymatic activity. Using one cycle at 95° C. for 90 seconds, 55° C. for 60 seconds, and 72° C. for 120 seconds, 30 cycles of PCR were repeated and the final reaction was performed at 95° C. for 90 seconds, 55° C. for 60 seconds, and 72° C. for 5 minutes. 10 μL of aliquot was taken from the mixture obtained therefrom and was electrophoresed over 1% agarose gel at 100 V for 30 minutes. This analysis revealed that the differentiation-staged cells show the same CD122 and perforin expression pattern according to previously published results known in the art. More CD122 was expressed in mNK cells than in pNK cells. Furthermore, co-culturing of pNK cells with stromal cells resulted in greater expression of CD122. Perforin was expressed in mNK cells. The co-culturing of pNK cells with stromal cells increased the expression of the perforin in the mNK cells produced in the culture (FIG. 3).

(D) Measurement of Differentiation Levels with or without Co-Culturing with Mouse Stromal Cells

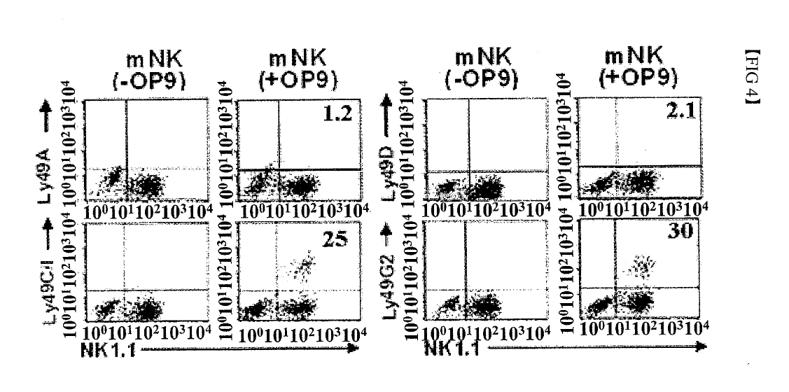

The expressions of cell-associated surface molecules (NK1.1 and Ly49) of mNK cells in the absence and presence of stromal cells (−OP9 and +OP9) were compared by FACS analysis as described in step (B) using NK1.1 and Ly49 antibodies (Pharmingen). The results demonstrated that mNK cells developed under co-culturing with stromal cells in the presence of IL-15 expressed more NK1.1 and Ly49 cell-associated surface molecules than those cells developed in the presence of IL-15 but without co-culturing with stromal cells. Therefore, stromal cells can improve and present specificity in the final differentiation stage of NK cells. The population of mNK cells developed under co-culturing with stromal cells were composed of 1.2% Ly49A⁺NK1.1⁺, 25% Ly49C/I⁺NK1.1⁺, 2.1% Ly49D⁺NK1.1⁺, and 30% Ly49G2⁺NK1.1⁺. However, the mNK cells developed without stromal cells failed to produce a cell population with the same composition (FIG. 4).

(E) Identification of Substances Capable of Inducing Mouse mNK Cell Differentiation by SAGE

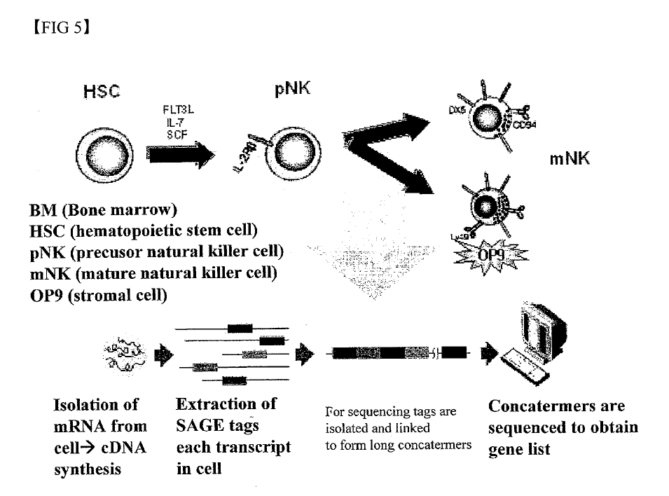

To identify the genes that are specifically expressed along each differentiation stage for the isolated HCSs, pNK cells and the two mNK cell populations developed by culturing with or without stromal cells, serial analysis of gene expression (SAGE) was performed (FIG. 5). In accordance with the same method as described in step (C), total RNAs were extracted from the NK development stage-specific cells including HSCs, pNK cells, and two populations of mNK cells. The mRNA was purified from 5 μg of total RNA with oligo (dT)₂₅ beads (Dynal A.S.) according to the manufacturer's instructions. cDNA was synthesized with a cDNA synthesis kit (Life Technologies) using 5′-biotinylated and 3′-anchored oligo (dT) primer as described in the manufacturer's protocol. SAGE tags were constructed from the cDNA and concatenated by T4 DNA ligase. The concatemers were cloned into Sph I-cleaved pZero-1 vector (Invitrogen) using T4 DNA ligase (Roche). After selection of positive colonies by PCR using M13 forward and M13 reverse primers, PCR products were obtained in accordance with PCR performed in the above step (C). The PCR products were sequenced with the Big-Dye sequencing kit and ABI377 sequencer (Perkin-Elmer Applied Biosystems, Branchburg, N.J.). The tag sequences were extracted with SAGE 300 software. A reference SAGE-tag database was constructed from the UniGene mouse database representing most of the known mouse expressed sequences in GenBank. The conditions used for determining SAGE tags in sequences included (i) the orientation of each transcript, (ii) the presence of a poly (A) signal (AATAAA or ATTAAA), (iii) the presence of a poly (A) tail, and (iv) the presence of the CATG cleavage site in the sequence terminal. All SAGE tags extracted from the reference sequences were used for building the reference SAGE database. A computational program, GIST (Gene Identification and Sequence Topography), was developed for matching experimental SAGE tags against the reference SAGE database for identifying potential corresponding genes for each SAGE tag. This revealed that among genes expressed in pNK cells but not expressed in any other development stages, Axl gene was expressed in a greater quantity and specifically in pNK cells (Table 1). For statistical analysis of data, p values were analyzed using a paired Student t test software program (Statview 5.1; Abacus Concepts, Berkeley, Calif., USA). The statistical significance (p) in the evaluation of differential expression of SAGE tags among the samples was determined by IDEG6 analysis using the v2, Audic, and Claverie methods (http://telethon.bio.unipd.it/bioinfo/IDEG6_form).

TABLE 1
GENE SPECIFICALLY EXPRESSED IN PNK CELLS

| Gene name | HSC | pNK | mNK(−OP9) | mNK(+OP9) | Unigene Id |
|---|---|---|---|---|---|
| Axl | 0 | 189 | 0 | 0 | Mm.4128 |

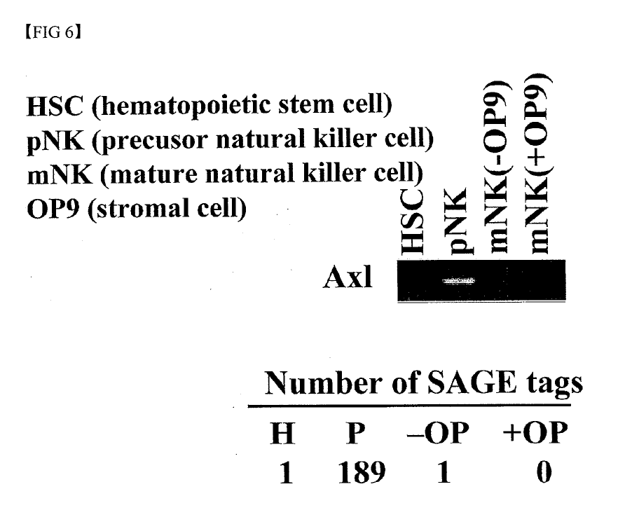

RT-PCR was conducted to confirm whether the Axl gene expression in pNK cells is substantially consistent with the result of SAGE. As a result, it is found that the number of SAGE tag was consistent with the expression pattern of Axl. This shows that the amount of the Axl expression was augmented in pNK cells (FIG. 6).

### EXAMPLE 2

### Effect of Axl Polyclonal Antibody on the Differentiation of Natural Killer Cell

(A) Expression of Mature Natural Killer Cell-Associated Receptors by Axl Polyclonal Antibody: Co-Culturing with Stromal Cells

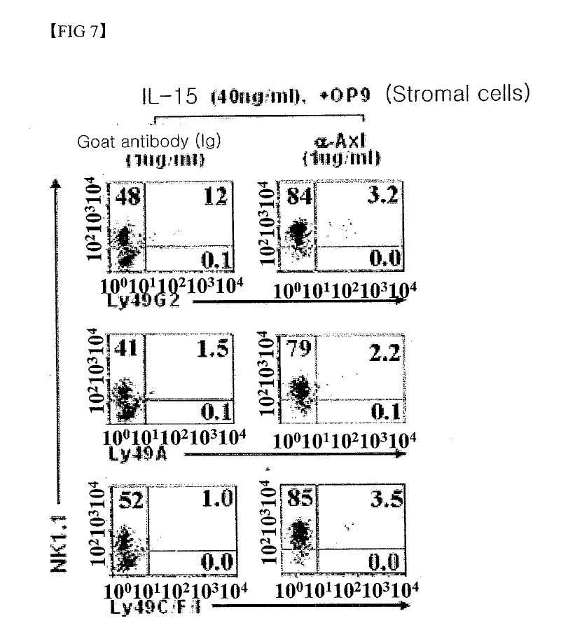

During the differentiation from mouse hematopoietic stem cells to natural killer cells, precursor natural killer cells (on day 7) were treated with 1 μg of Axl polyclonal antibody (Santa Cruz Biotechnology, Inc. sc-1096) and, in accordance with the same method as described in Example 1, the pNK were co-cultured with stromal cells in the presence of IL-15 (40 ng/mL) for 6 days. The developed natural killer cells were stained with NK1.1 antibody and antibodies directed against natural killer cell-associated receptors (Ly49G2, Ly49A, Ly49C/F/I, Pharmingen) and were analyzed by FACS in accordance with the same method as described in Example 1(B). The test cells treated with Axl polyclonal antibody were compared with control cells treated either with goat Ig (R&D) or with no antibody at all. All cell populations were analyzed by FACS according to the method described in Example 1(B). As a result, it was found that, in cells treated with Axl polyclonal antibody, the level of differentiation was increased from 1.2% to 3.2% in Ly49A⁺NK1.1⁺ cell, 1% to 3.5% in Ly49C/I⁺NK1.1⁺ cell, and 1.2% to 3.2% in Ly49G⁺NK1.1⁺ cell, respectively. This demonstrates that Axl polyclonal antibody augments the development of mature natural killer cells by about two times and positively affects the differentiation from pNK cells to mNK cells (FIG. 7).

(B) Expression of Mature Natural Killer Cell-Associated Genes by Axl Polyclonal Antibody: Co-Culturing with Stromal Cells

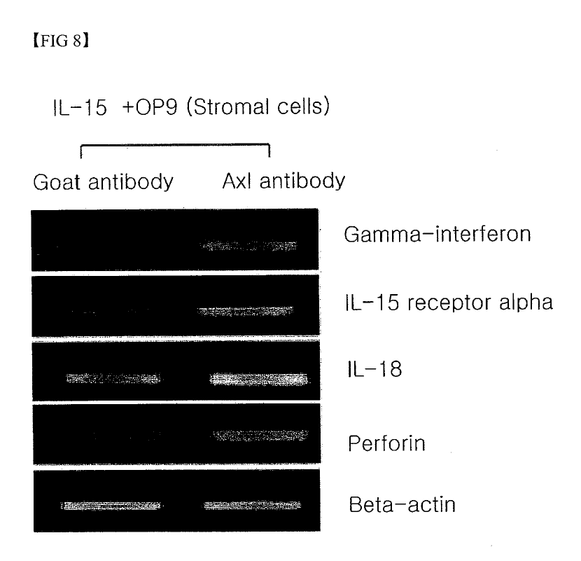

The expression of mature natural killer cell-associated genes (interferon-γ, 5′-agcggctgactgaactcagattg-3′ (SEQ ID NO: 7) and 5′-gcacagttttcagctctatagg-3′ (SEQ ID NO: 8); IL-15Ra, 5′-ccaacatggcctcgccgcagct-3′ (SEQ ID NO: 9) and 5′-ttgtagagaaagcttctggctc-3′ (SEQ ID NO: 10); IL-18, 5′-aggtacaaccgcagtaatgcgg-3′ (SEQ ID NO: 11) and 5′-agtgaacattacagatttatccc-3′ (SEQ ID NO: 12); and perforin, 5′-gtcacgtcgaagtacttggtg-3′ (SEQ ID NO: 13) and 5′-aaccagccacatagcacacat-3′ (SEQ ID NO: 14) in the same natural killer cells as described in the above (A) was analyzed by the PCR as described in the above Example 1(C). As a result, the test cells treated with Axl polyclonal antibody show an increase in the expression of the mNK cell-associated genes as compared to the control cell population treated with goat antibody. This demonstrates that the Axl polyclonal antibody positively affects the differentiation of pNK cells into completely mature natural killer cells (FIG. 8).

(C) Expression of Mature Natural Killer Cell-Associated Receptors by Axl Polyclonal Antibody: Culturing without Stromal Cells

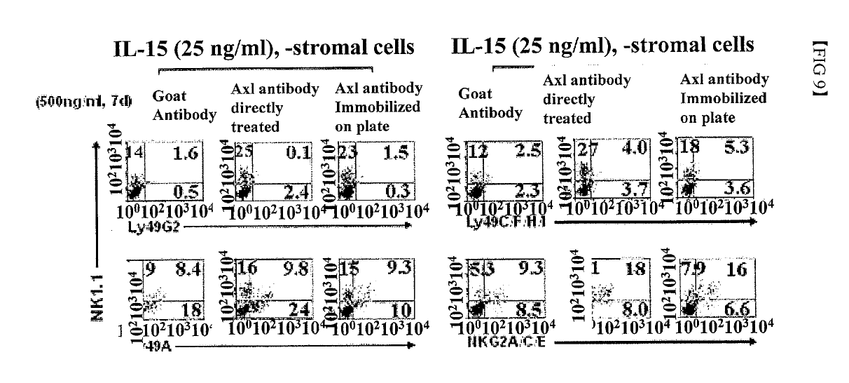

In order to confirm the effect of Axl polyclonal antibody on the development of mature natural killer cells under different conditions, pNK cells were cultured with 25 ng/mL of IL-15 and 500 ng/mL of Axl polyclonal antibody but without co-culturing with stromal cells, while being fixed onto the culture dish. The developed cells were stained with NK1.1 antibody and antibodies directed against natural killer cell-associated receptors (Ly49G2, Ly49A, Ly49C/F/I, NKG2A/C/E) and were analyzed by FACS in accordance with the same method as described in Example 1(B). As results, compared to cells not treated with Axl polyclonal antibody, it is found in the cells treated with the antibody that the differentiation increased from 8.44% to 9.8% and 9.3% in Ly49A⁺NK1.1⁺ cell, from 2.5% to 4% and 5.3% in Ly49C/F/I⁺NK1.1⁺ cell, and from 9.3% to 18% and 16% in NKG2A/C/E+NK1.1⁺ cell, respectively (FIG. 9).

(D) Expression of Mature Natural Killer Cell-Associated Genes by Axl Polyclonal Antibody: Culturing without Stromal Cells

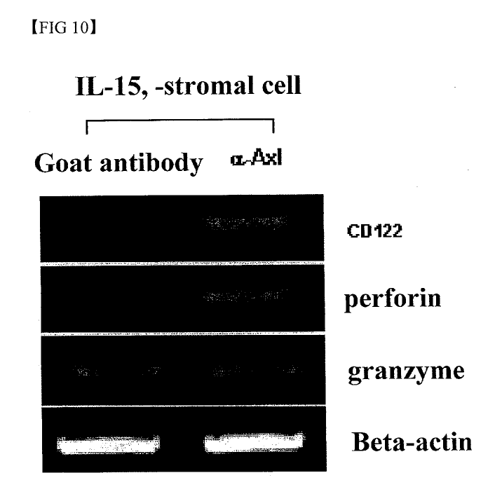

The expression of mature natural killer cell-associated genes (CD122, perforin, and granzyme B) in the same natural killer cells as described in the above (C) was analyzed by the RT-PCR as described in the above Example 1(C). As a result, it was found that in cells treated with Axl polyclonal antibody, the expression of mNK cell-associated genes was increased compared to the control population of cells treated with goat antibody. This demonstrates that the Axl polyclonal antibody positively affects the differentiation into completely mature natural killer cells even in the absence of stromal cells (FIG. 10).

### EXAMPLE 3

### Effect of Axl Polyclonal Antibody on Interferon-γ Production by Natural Killer Cells

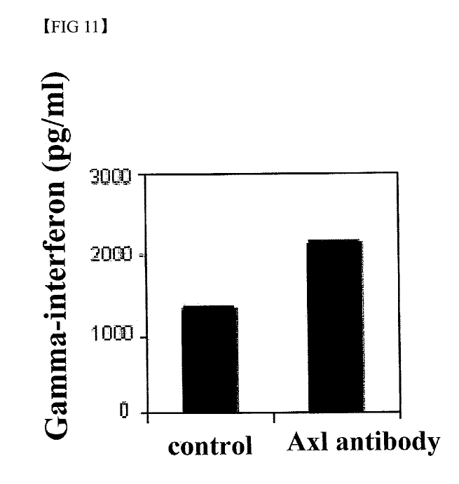

The pNK cells obtained as described in Example 1 were co-cultured with stromal cells in the presence of Axl polyclonal antibody (500 ng/mL) and IL-15 (10 ng/mL) to produce mature natural killer cells, which were then activated with the treatment of IL-2 (10 ng/mL, R&D). After 24 hours, the amount of the produced interferon-γ was measured using interferon-γ ELISA kit (BD Pharmingen). As a result, it was shown that the cells treated with Axl polyclonal antibody increased the amount of interferon-γ produced in the mNK cells compared to the control cell population treated with goat antibody (FIG. 11).

### EXAMPLE 4

### Effect of Axl Polyclonal Antibody on the Proliferation of pNK Cells

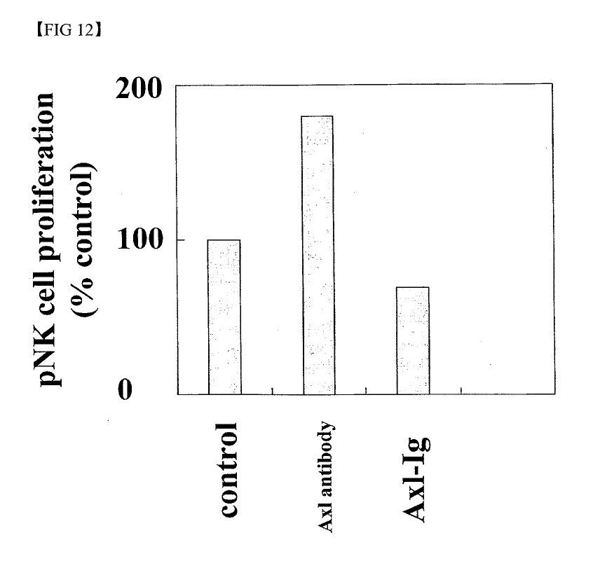

Stromal cells were plated in 96-well microtiter plate. After one day, pNK cells obtained in the above Example 1(B) were seeded to 2.5×10⁴ cells/well and were treated with IL-15 (25 ng/mL) along with 500 ng/mL of goat antibody, Axl polyclonal antibody (α-Axl) or Axl-Ig. After 48 hours, the proliferation of pNK cells was analyzed by MTS assay (CellTiter 96 Aqueous Assay, Promega, Madison, Wis.). The MTS is a calorimetric assay using tetrazolium compound [3-(4,5-dimethylthiazol-2-yl)-5-(3-carboxymethoxy-phenyl)-2-(4-sulfophenyl)-2H-tetrazolium, inner salt; MTS] and electron-coupling reagent phenazine methosulfate (PMS). On the last day of cell culture, 0.025 mL of MTS/PMS mixture solution was added to each well and was incubated for 30 minutes. Then, optical absorbance was measured at the wavelength of 495 nm using ELISA Reader (Molecular Devices Co., Sunnyvale, Calif., USA). It was found that the proliferation of pNK cells treated with Axl polyclonal antibody is about twice as much as that of the control cell population. In the meantime, the proliferation of pNK cells treated with Axl-Ig was reduced. This demonstrates that the transfer of Axl signal by Axl polyclonal antibody has a significant effect on the proliferation of pNK cells (FIG. 12).

### EXAMPLE 5

### Effect of the Interaction of Axl and its Ligand Gas6 on the Differentiation of Natural Killer Cells

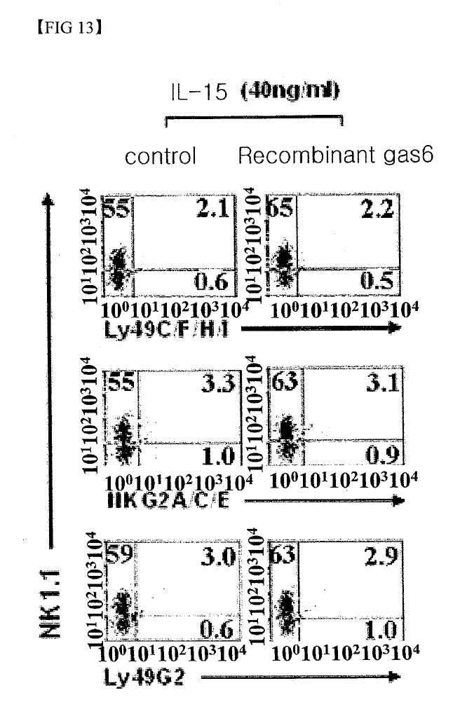

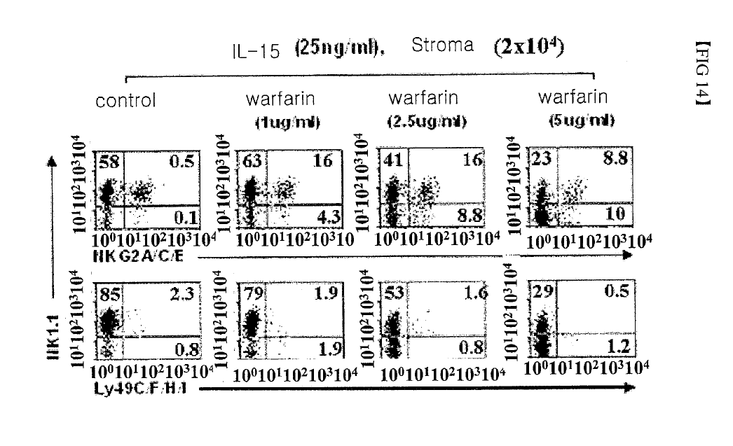

In order to study the effect of the signal transfer by the binding between Gas6 and Axl on the differentiation of natural killer cells, mouse recombinant Gas6 (500 ng/mL, R&D) was added during the differentiation. However, it did not affect the differentiation of natural killer cells (FIG. 13). Therefore, further experiments were conducted to determine if the biological activity of Gas6 is affected by γ-carboxylation. For the next set of experiments, Gas6 expressed in stromal cells was used. The effect of Gas6 on the signal transfer through Axl was examined by using warfarin (Sigma), which selectively inhibits the post-translational γ-carboxylation of Gas6. The pNK cells differentiated from HSCs were treated with IL-15 (25 ng/mL) along with 1 μg/mL, 2.5 μg/mL, and 5 μg/mL of warfarin and were co-cultured with stromal cells (2×10⁴) for 7 days. The level of differentiation from pNK cells to mNK cells was analyzed by FACS as described in the above Example 1(B). As a result, the quantity of receptors specifically expressed in NK1.1, NKG2A/C/E and Ly49C/F/H/I natural killer cells was decreased in a concentration-dependent manner (25%, 16%, 16%, and 8.8% for NK1.1⁺NKG2A/C/E⁺; and 2.3%, 1.9%, 1.6%, and 0.5% for NK1.1⁺Ly49C/F/H/I⁺) (FIG. 14). This demonstrates that the interaction between Axl and its ligand Gas6 plays a role in the differentiation from pNK cells to mNK cells.

### EXAMPLE 6

### Construction of Mouse Gas6 Expression Vector and Retrovirus Vector

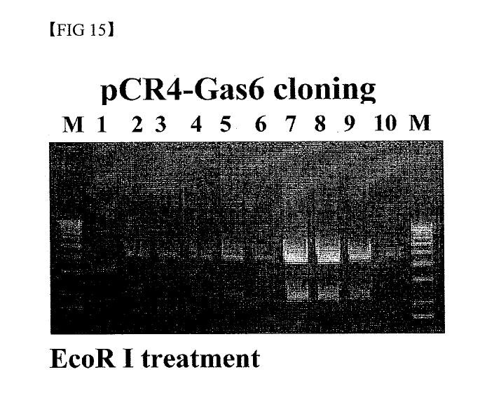

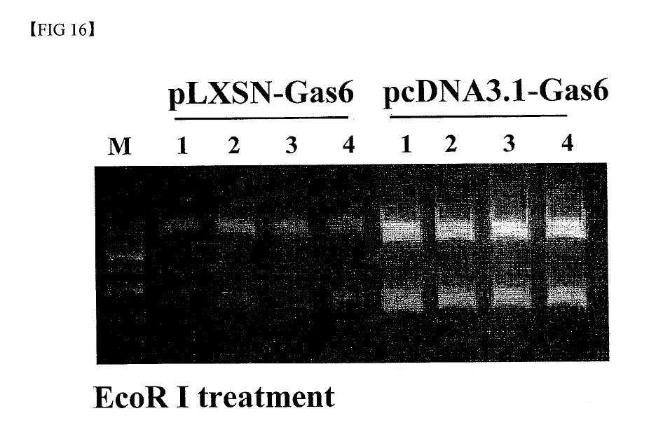

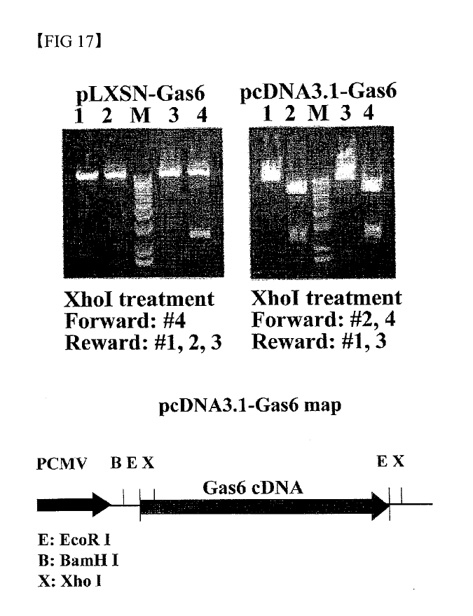

To produce active Gas6, RT-PCR was performed using the RNAs extracted from mouse stromal cells and Gas6 primers (5′-ggcctcgagcatgccgccaccgcccgggc-3′ (SEQ ID NO: 15) and 5′-ggcgaattccggtctagggggtggcatgc-3′ (SEQ ID NO: 16) as described in Example 1(C) to amplify Gas6 cDNA (100 ng), which was cloned into pCR4-TOPO (50 ng, Invitrogen) digested with restriction enzyme EcoRI (1 U, Roche) (FIG. 15). After the clones were sequenced, the cDNA of the same clone as reference sequence (gene ID; AK086187) was used to construct mouse Gas6 expression vector and retrovirus vector. Retrovirus vector pLXSN (Invitrogen) was digested with EcoRI (1 U). To this was added mouse Gas6 cDNA (100 ng) which had been isolated by digesting pCR4-Gas6 #8 with EcoRI (1 U). Then, T4 DNA ligase was treated to construct a recombinant pLXSN-Gas6 (FIG. 16, left). pcDNA3.1(+) was digested with EcoRI (1 U) and CIP(calf intestinal alkaline phosphatase). To this was added mouse Gas6 cDNA (100 ng) which had been obtained by treating pCR4-Gas6 #8 with EcoRI (1 U). The mixture was reacted with T4 DNA ligase (1 U) to construct a recombinant pcDNA3.1-Gas6 (FIG. 16, Right). In these constructed expression vectors, the direction of mouse Gas6 cDNA relative to the virus and CMV promoter was confirmed by using XhoI (1 U, Roche). The clones in which the cDNA was forwardly and reversely positioned to the promoter were designated as pLXSN-Gas6/F, pLXSN-Gas6/R and pcDNA3.1-Gas6/F, pcDNA3.1-Gas6/R, respectively (FIG. 17).

### EXAMPLE 7

### Preparation of Mouse Gas6 Transfectant

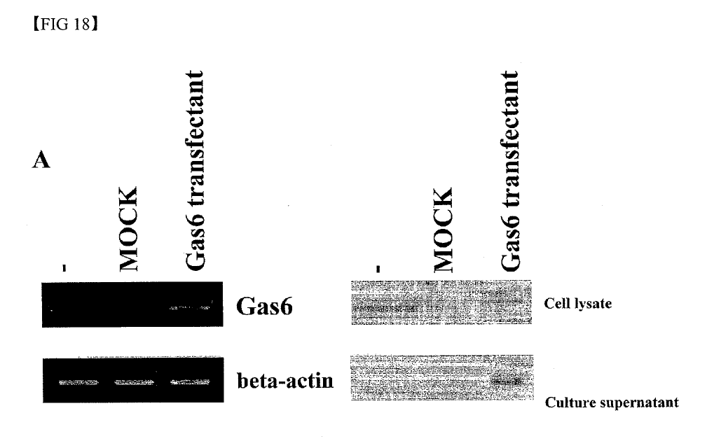

NIH3T3 cell line (ATCC) which does not express Gas6 gene was transfected with pcDNA3.1 (+), pcDNA3.1-Gas6/F and pcDNA3.1-Gas6/R using lipofectamine (Invitrogen). The transfectants were screened on the medium containing antibiotics G418 and were limitedly diluted to give the clones having the mouse Gas6 gene overexpressed. In the meantime, for the construction of mouse Gas6-expressing retrovirus, Gas6 retrovirus vectors pLXSN-Gas6/F and pLXSN-Gas6/R were transfected into PT67 cell line. The transfectants were screened on antibiotics G418-containing medium and were limitedly diluted to give retrovirus-infected NIH3T3 cell line. The expression level of Gas6 was confirmed by performing the same RT-PCR (A) as described in the above Example 1(C) using Gas6 primers (5′-ggcctcgagcatgccgccaccgcccgggc-3′ (SEQ ID NO: 17) and 5′-ggcgaattccggtctagggggtggcatgc-3′ (SEQ ID NO: 18) and Western blot analysis (FIG. 18(B)). The Western blot analysis was carried out as follows. The cells were collected from a culture dish and were treated with lysis buffer (20 mM HEPES pH 7.9, 100 mM KCl, 300 mM NaCl, 10 mM EDTA, 0.5% Nonidet P-40, 1 mM Na₃VO₄, 1 mM PMSF, 100 mg/mL aprotinin and 1 mg/mL leupeptin). The lysate was stored on ice for 30 minutes. The protein concentration was measured using Bradford reagent (Bio-Rad, Hercules, Calif.). Equal amounts of protein lysate were electrophoresed over 10% SDA-PAGE and then the electrophoresed proteins were transferred to PVDF membrane (Millipore, Marlborough, Mass.). The PVDF membrane was reacted with a blocking buffer solution (1% BSA and 5% skim milk in PBS) at the temperature of 4° C. overnight, and then washed three times with TBST (50 mM Tris. pH 7.4, 150 mM NaCl, 0.05% Tween 20). The PVDF membrane containing the protein was treated first with goat-anti-mouse Gas6 antibody (Santa Cruz) at room temperature for 1 hr and then treated with HRP-conjugated anti-goat IgG (Santa Cruz) at room temperature for another 1 hr. After washing three times the membrane with TBS, signal detection was carried out using ECL system (Amersham-Pharmacia Biotech, Arlington Height, Ill.).

### EXAMPLE 8

### Effect of Murine Gas6 on the Differentiation of Natural Killer Cells

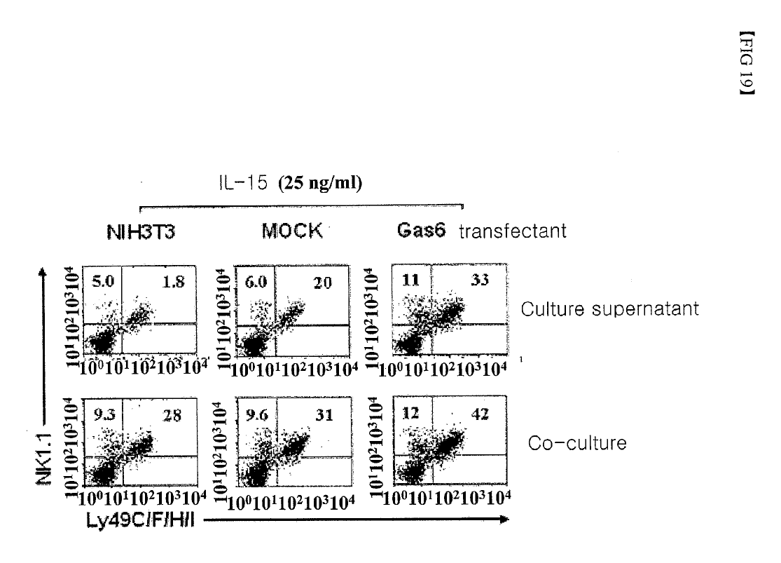

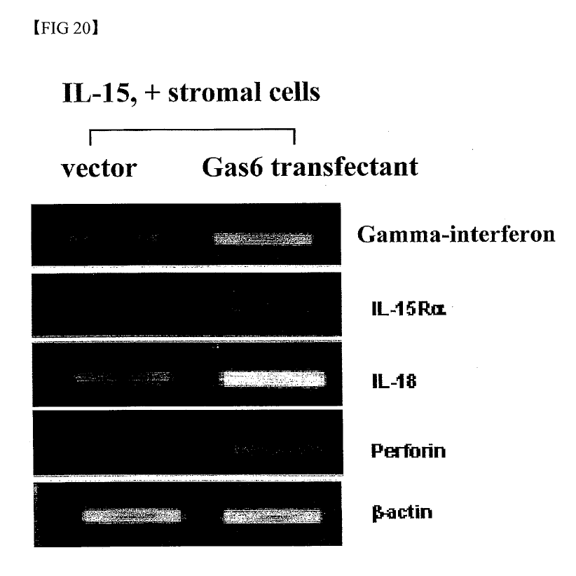

HSC cells, that are Lin− and c-kit+ and isolated from mouse bone marrow, were cultured in 24-well plate at the concentration of 1×10⁶/mL with RPMI medium containing SCF (30 ng/mL), IL-7 (0.5 ng/mL), Flt3L (50 ng/mL), indometacine (2 μg/mL), and gentamicin (2 μg/mL). After culturing for 6 days under the condition of 37° C., 5% CO₂, the resulting pNK cells were cultured with IL-15 (20 ng/ml) together with murine Gas6 transfectant for 24 hrs. The resulting mixture was centrifuged for 10 min at 2000 rpm and the supernatant culture sample was again cultured for another 6 days with murine Gas 6 transfectant and Axl polyclonal antibody for the differentiation into mNK cells. The resulting cells were harvested and tested for the expression of mNK surface molecules using the FACS method described in Example 1(B). Purity of the cells at each stage was all at least 95% based on FACS analysis. In order to confirm whether murine Gas6 protein secreted from the murine Gas6 transfectant affects the differentiation of pNK cells into mNK cells, the degree of differentiation of NK cells was measured in different cell populations. The control groups contained pNK cells either co-cultured with native NIH3T3 cells or NIH3T3 cells mock-transfected with empty vector (pcDNA3.1(+)). Alternatively, the control groups were cultured in the culture medium (or culture supernatant) obtained from a culture of either native NIH3T3 cells or NIH3T3 cells mock-transfected with empty vector (pcDNA3.1(+)) diluted in a 1:20 ratio, The experimental group contained pNK cells co-cultured with the transfectant that over-expresses murine Gas6 protein, or alternatively, with the culture supernatant obtained from a culture of said transfectant diluted in a 1:20 ratio. As a result, it was found that, pNK cells co-cultured with murine Gas6 transfectant have greater degree of differentiation as compared to that of the control groups (i.e., increased from about 14% to 47%, see FIG. 19). In addition, when population of cells cultured in the culture supernatant obtained from the Gas6 transfectant (5×10⁶) was compared to that of cells cultured in the culture supernatant fron non-Gas6 expressing transfectants, the expression levels of perforin, IL-18 and interferon-γ in mNK cells were found to be greater in the cells cultured in the Gas6 transfectant culture supernatant (FIG. 20). Expression levels of the genes were measured according to the method described in Example 1(C). Therefore, these experiments confirmed that, in conjunction with Gas6 protein, Axl exerts a significant effect on the differentiation of HCS cells into NK cells via signal transfer.

### EXAMPLE 9

### Effect of Gas6 on the Production of Interferon-γ by NK Cells

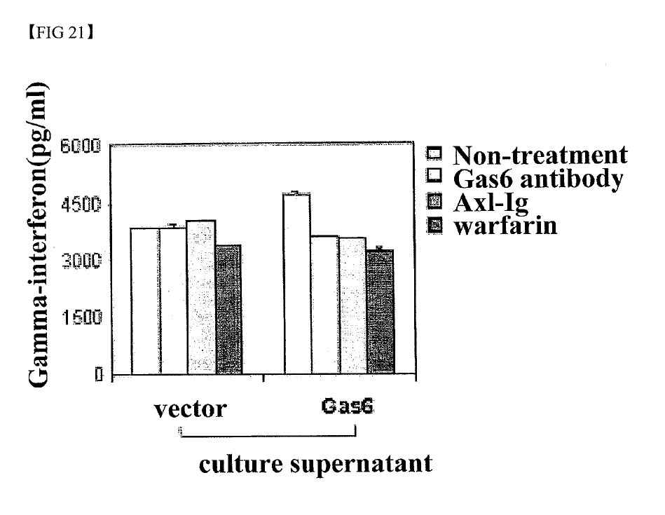

pNK cells obtained as described in Example 1 were co-cultured with IL-15 (10 ng/mL) and stromal cells. In addition, Gas6 antibody (500 ng/mL), Axl-Ig (500 ng/ml) and warfarin (500 ng/ml) were separately added to the pNK cells thus obtained. The pNK populations were then differentiated into mNK cells by culturing them in either a 1:20 dilution of the supernatant of Gas6 transfectant or in a 1:20 dilution of the supernatant of vector-only-transfectant. The differentiated cell polpulations were then treated with human IL-2 (10 ng/ml, R&D). After 24 hrs, the amount of interferon-γ produced by the differentiated cell populations was determined with the interferon-γ ELISA kit (BD Pharmingen). As a result, it was found that there is an increase in interferon-γ produced in the group to which the supernatant of Gas6 transfectant was added. For the Gas6 supernatant treated populations, it was found that the amount of interferon-γ production was decreased in populations treated with Gas6 antibody (500 ng/mL), Axl-Ig (500 ng/ml) and warfarin (500 ng/ml) as compared to the non-Gas6/Axl-Ig/warfarin-treated population. Thus, these experiments confirmed that Gas6 signal is mediated by binding of Gas6 to Axl protein and is therefore involved in the activation of NK cells (FIG. 21).

### EXAMPLE 10

### Effect of Gas6 on the Proliferation of pNK Cells

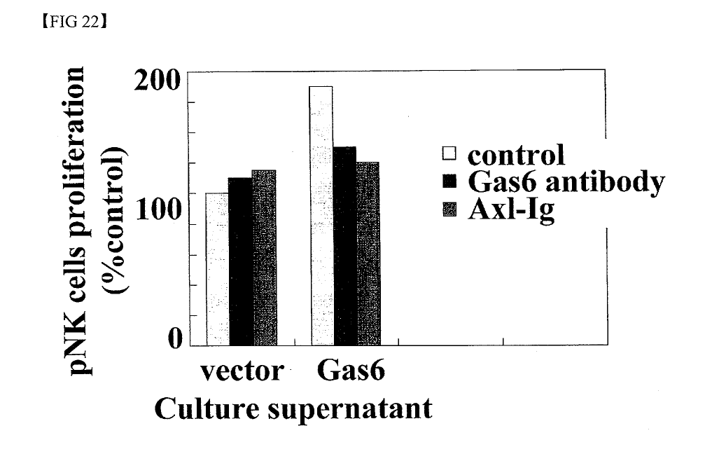

Stromal cells were plated in 96-well microtiter plate. After one day, pNK cells obtained according to Example 1 were seeded to 2.5×10⁴ cells/well. In addition to being cultured in IL-15 (25 ng/mL), the cells were treated with either 500 ng/ml of Axl-Ig or Gas6 antibody (a-Gas6). Furthermore, the treated cells were cultured either in Gas6 culture supernatant or vector culture supernatant as described in Example 8. After 48 hrs, the proliferation of pNK cells was analyzed by MTS assay as described in Example 4. Compared to the control group treated with vector culture supernatant, the group treated with the Gas6 culture supernatant exhibited an increase in pNK proliferation. The increase in pNK proliferation observed in the Gas6 supernatant-cultured populations was reduced in the presence of either Gas6 antibody or Axl-Ig in the culture. Further, considering that the proliferation of pNK cells is reduced by the presence of either Axl-Ig or an antibody directed against Gas6, the experiments confirm that the proliferation of pNK cells is induced by the interaction between Axl and Gas proteins (FIG. 22).

### EXAMPLE 11

### Construction of Murine Axl Expression System

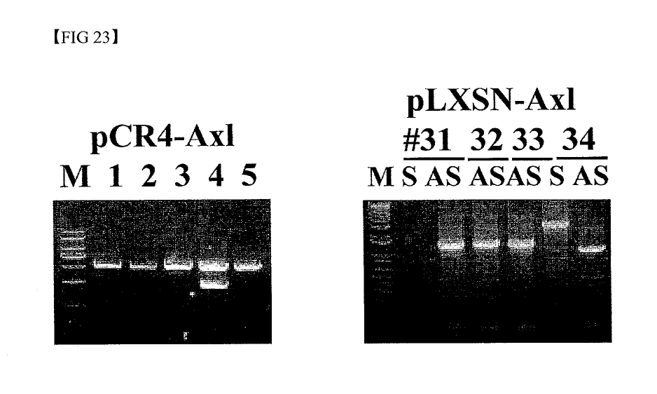

Total RNA isolated from murine RAW264.7 macrophage, which highly expresses Axl protein, was performed using Axl primers (sense; 5′-ggtgcccatcaacttcggaa (SEQ ID NO: 19), antisense; 5′-ggatgtcccaggtggaagatt (SEQ ID NO: 20)) according to the RT-PCR protocol described in Example 1(C). A 2,750-bp murine Axl cDNA (100 ng) product was cloned into pCR4-TOPO (50 ng) that was digested with EcoRI(1 U) (FIG. 23). After digesting retrovirus vector pLXSN with the restriction enzymes of EcoRI(1 U) and CIP(1 U), Axl cDNA (100 ng) obtained from the treatment of pCR4-Axl #4 with EcoRI restriction enzyme was added and T4 DNA ligase (1 U) was added. Recombinant pLXSN-Axl was thus obtained. The direction of Gas6 cDNA relative to the promoter in the retrovirus vector was confirmed by PCR reaction using the primer downstream of the cloning site and the Axl primer and by base sequencing method. The clones in which the cDNA was forwardly and reversely positioned relative to the promoter were designated as pLXSN-Axl/F and pLXSN-Axl/R, respectively.

### EXAMPLE 12

### Production of Murine Axl-IgG Fusion Protein

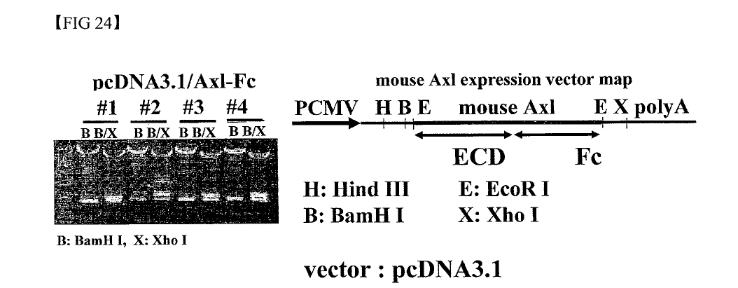

Transfection of PT67 cell line was carried out using the same method as described for the production of the retrovirus expressing Gas6 protein in Example 7. In order to prepare a cell line expressing Axl-IgG fusion protein, at the 5′ and 3′ ends of murine IgG1 Fc (constant fragment) BamHI and XhoI linkers were introduced respectively. PCR was then carried out according to the method described in Example 1(C) for the amplification of murine Axl-IgG1 Fc, and the PCR product was cloned into pCR4-TOPO (pCR4-Fc). Meantime, at the 3′ end of extracellular domain (ECD) of Axl gene, a BamHI linker was introduced, and the Axl ECD gene was amplified by PCR and cloned into pCR4-TOPO (pCR4-Axl/ECD). pCR4-Fc was treated with Xho I(1 U) and BamHI(1 U, Roche) to separate the Fc fragment of 820 bp. pCR4-Axl/ECD was treated with EcoRI(1 U) and BamHI(1 U), and the resulting 1350 bp Axl/ECD (100 ng) and the above obtained Fc fragment were cloned into the EcoRI-XhoI site of pcDNA3.1(50 ng). DNA clones were then prepared using the restriction enzymes of BamHI(1 U), BamHI(1 U)/XhoI(1 U) (FIG. 24). Finally, 293T cells were transfected with plasmid pcDNA3.1/Axl-Fc, the transfectants were selected from G418 containing culture media, and the clones which over-express Axl-IgG fusion protein was obtained by limiting dilution method.

### EXAMPLE 13

### Effect of Murine Axl-IgG Fusion Protein on the Differentiation of NK Cells

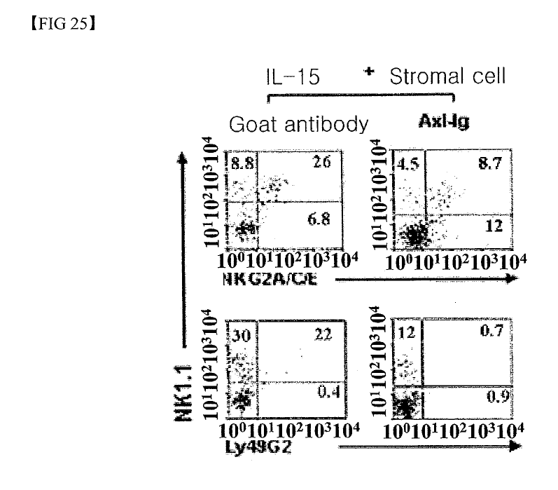

In order to determine the effect of blocking the signal transfer by Axl on the development of natural killer cells, IL-15 (25 ng/ml) and Axl-Ig were added while the differentiated pNK cells were being co-cultured with stromal cells (2×10⁴) for seven days. Subsequently, the level of differentiation of the pNK cells into mNK cells was determined by FACS analysis following staining with antibodies of NK1.1, NKG2A/C/E, and Ly49G2. As a result it was observed that, when the action of the Axl-Gas6 complex was blocked by the addition of Axl-Ig, the number of cells positive for NK1.1, NKG2A/C/E and Ly49G2, which are markers of mNK cells, was reduced. Thus, it is found that Axl-Gas6 interaction plays an important role in the differentiation of pNK cells into mNK cells (FIG. 25).

### EXAMPLE 14

### Differentiation of NK Cells by Inhibiting Axl Protein Expression

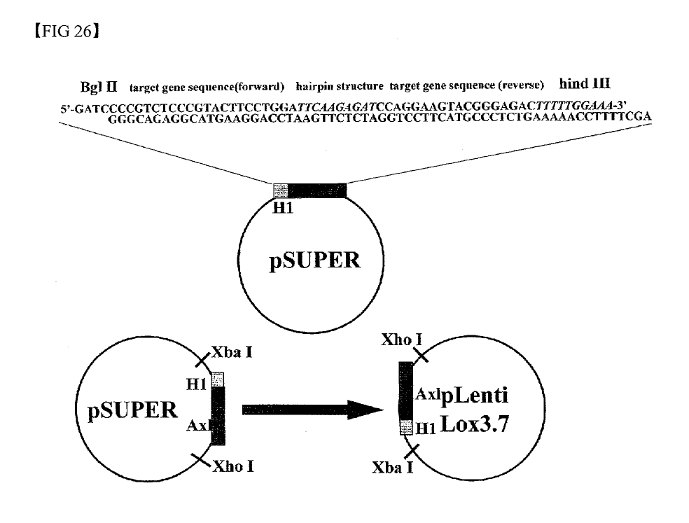

Lenti virus vector was used to remove Axl from NK cells (FIG. 26).

(1) Preparation Of siRNA

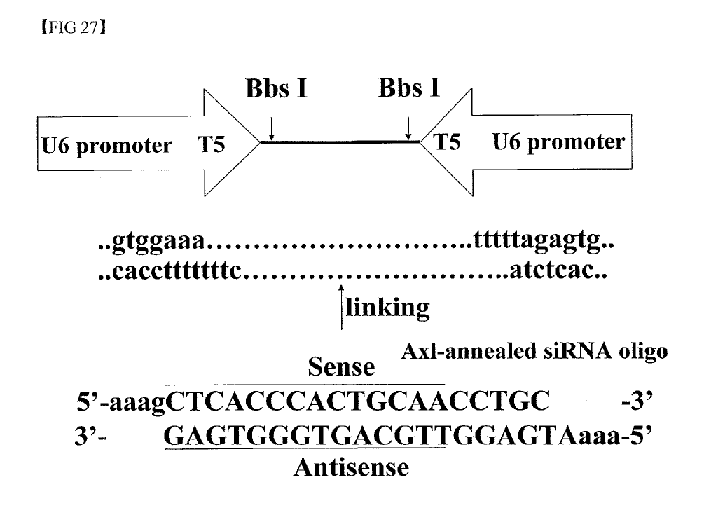

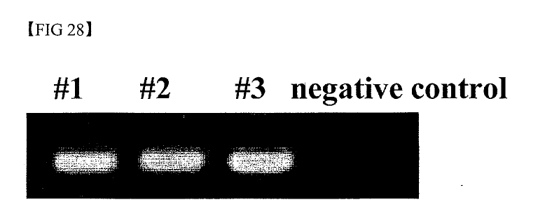

To selectively block the site with Axl activity, an oligonucleotide which can remove the function of Axl gene was prepared as follows. A target base sequence for blocking was set as gtctcccgtacttcctgga (#1) (SEQ ID NO: 21), ctcacccactgcaacctgc (#2) (SEQ ID NO: 22), agacctacacagtttcctc (#3) (SEQ ID NO: 23). For said three types of base sequence, one primer which comprises bases of aaag overhanging at 5 end in a sense direction and the other primer which comprises bases of aaaa overhanging at 3 end in an antisense direction were prepared, respectively. Each of these oligomers were annealed at 95° C. to prepare a double strand, while double promoter pFIV-H1/U6 siRNA-GFP was digested with BbsI(Roche), and then purified and cloned (FIG. 27). Among the clones obtained after the transfection, PCR was carried out using U6 PCR primer and anti-sense siRNA oligonucleotide in order to find out the clones containing siRNA sequence. As a result, for the clones containing siRNA sequence, a PCR product of about 100 bp was confirmed (FIG. 28).

(2) Establishment Of Lenti Virus siRNA Expression System

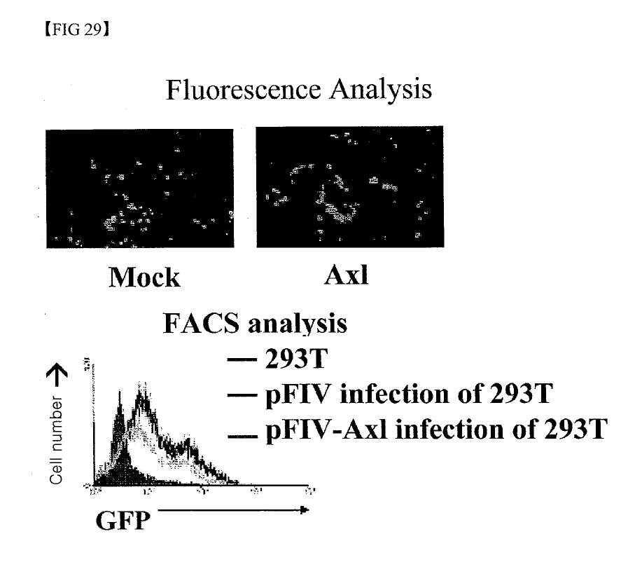

For preparing Lenti virus which can transfect NK cells, Lenti virus vector expressing siRNA and helper vectors of VSVG, RSV-REV, and pMDL g/pRRE were used. 292T cells (ATCC) which can form virus constructs were added to 10² cm cell culture dish in an amount of about 5×10⁵ cells. After about 24 hrs, the cells were transfected with the vectors prepared above, each with 1.5 μg of lipofectamine. New DMEM culture medium was supplied about 4 hrs later, and culturing was continued in CO₂ incubator at 37° C. About 48 hrs later, the culture media was centrifuged for 5 min at 3000 rpm and the cellular debris was removed. Culture supernatant was filtered through Millex-HV 0.45 μm PVDF filter system and stored. For the confirmation of the virus production from thus obtained culture supernatant, 293T cells were seeded to 2.5×10⁴ cells/well of 6-well culture dish. After 24 hrs of culturing, virus particles were mixed with DMEM media containing 10% fetal bovine serum in 1:1 ratio and were treated into 293T cells. At this moment, in order to increase the degree of binding of virus capsid to the cellular membrane, 8 μg/ml of polybrene was further added. After the transfection with the virus for 24 hrs, the media was replaced with new DMEM media containing 10% fetal bovine serum. After another 48 hrs of culturing, fluorescence spectroscopy and FACS analysis as described in Example 1(B) were carried out. As a result, it was confirmed that the cells were successfully transduced with pFIV expression vector tagged with GFP (FIG. 29).

(3) Transfection with Lenti Virus During the Differentiation of Hematopoietic Cells into Natural Killer Cells

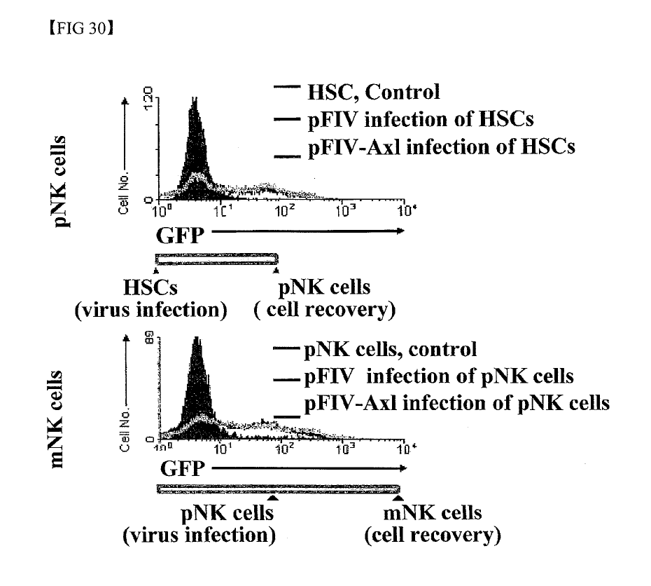

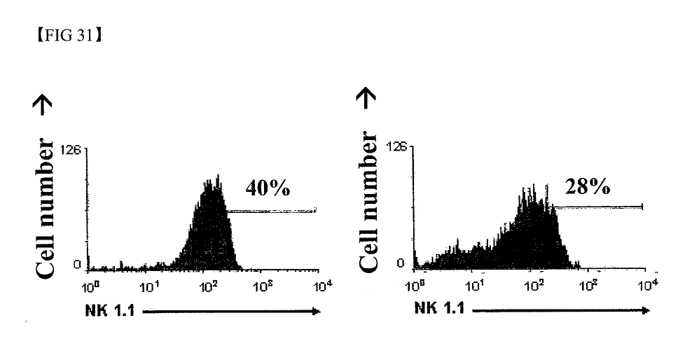

Hematopoietic cells were isolated from mouse bone marrow. Virus particles were added to the cells in an amount of about 1×10⁶ particles per cell and the transfection was carried out for 24 hrs. After culturing the cells in the media comprising IL-7 (0.5 ng/ml), FLT3L (50 ng/ml) and SCF(30 ng/ml) for about 6 days, precursor NK cells were obtained. According to the FACS analysis as described in Example 1(B), GFP expression was increased thanks to good transfection by Lenti virus and at the same time, the differentiation of NK cells was inhibited of Axl expression (FIG. 30 and FIG. 31).

### EXAMPLE 15

### Determination of Inhibitory Activity of NK Cells on Tumorigenesis in a Cancerous Animal Model

For establishing a cancerous animal model, B16F10 melanoma cells which are cancer cells originating from C57BL/6 were injected intravenously to 7 to 8 week old C57BL/6 mice (5×10⁴ cells per animal). Next day, mNK cells differentiated by human IL-2(10 ng/ml, R&D) and a control antibody (1 g/ml), or by IL-2 (10 ng/ml) and Axl polyclonal antibody (1 μg/ml), respectively, were intravenously injected into the animal (1×10⁶ cells per animal). Two weeks later, migration of the cancer cells and anticancer efficacy were determined by measuring the number of B16F10 melanoma cells found in the animal's lung. As a result, the number of cancer colonies found in the lung tissues injected with mNK cells that have been stimulated by Axl polyclonal antibody was reduced compared to that of the mice injected with mNK cells treated with the control antibody (i.e., 4 to 10 fold decrease). However, for the lung tissues injected with mNK cells treated with Axl-Ig, number of cancer colonies were significantly increased, confirming that Axl can regulate not only the formation of cancer cells by activating NK cells but also the migration of the cancer cells.

### EXAMPLE 16

### Determination of Tumoricidal Activity of NK Cells in a Cancerous Animal Model

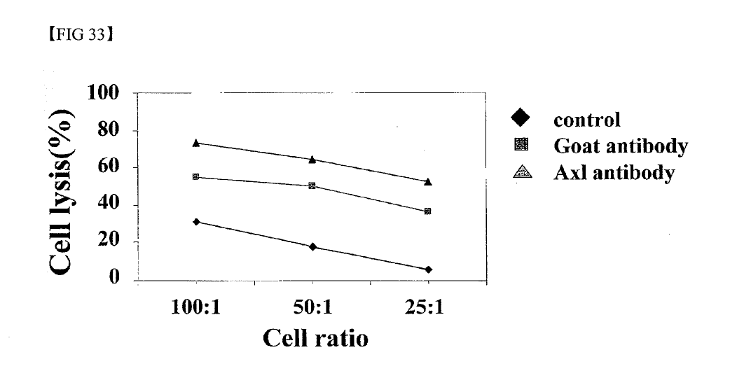

In order to compare tumoricidal activity of NK cells, spleen cells were separated from the mouse of Example 15, stimulated with human IL-2 (10 ng/ml) for 48 hrs, and treated into YCA-1 cells tagged with ⁵¹Cr (100 μci) for 2 hrs in ratio of 1:100, 1:50 and 1:25. After 4 hrs of incubation, the amount of ⁵¹Cr released to the culture supernatant was measured. As a result, tumoricidal activity was found for each group as follows; for the control group it was about 30%, for the test group in which the NK cells differentiated by treating with control antibody were injected it was 58%, and for the test group in which the NK cells differentiated by treating with Axl polyclonal antibody were injected it was about 75%, which is significantly higher than that of the control group (FIG. 33).

### EXAMPLE 17

### Survival Rate Analysis of Cancer-Induced Mouse after the Treatment with NK Cells in a Cancerous Animal Model

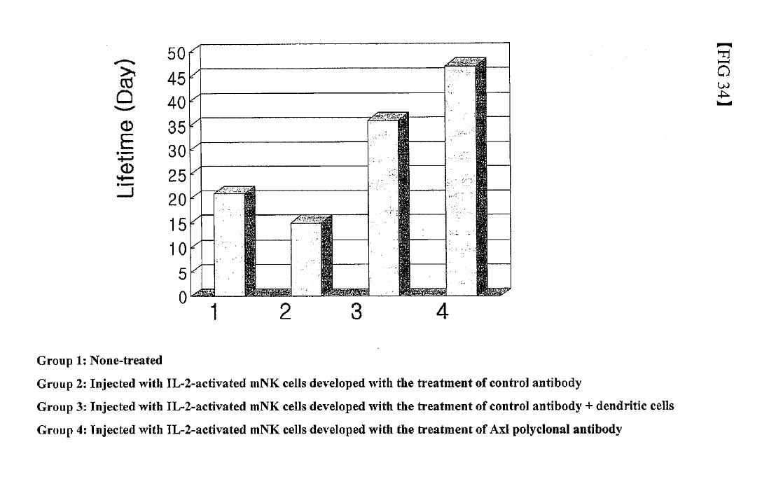

By subcutaneously injecting B16F10 melanoma cells which are cancer cells originating from C57BL/6 in an amount of 5×10⁴ cells per animal, cancer was induced in 7 to 8 week old C57BL/6 mice. For the first group of mice, no further substance was injected. For the second group, mNK cells differentiated by the treatment with a control antibody (1 μg/ml), and human IL-2 (10 ng/ml, R&D) were further subcutaneously injected. For the third group, mNK cells differentiated by the treatment with a control antibody (1 μg/ml) and human IL-2 (10 ng/ml, R&D) and dendritic cell were further subcutaneously injected. For the fourth group, mNK cells differentiated by the treatment with Axl polyclonal antibody (1 μg/ml) and human IL-2 (10 ng/ml, R&D) were further subcutaneously injected. The amount of the injected mNK cells was 1×10⁶ per animal. As a result, it was found that survival days of the mice were 21, 15, 36 and 47, respectively, for said four groups in order. Thus the mice treated with mNK cells differentiated by the treatment with Axl polyclonal antibody (1 μg/ml) and human IL-2 (10 ng/ml, R&D) survived the longest, and considering it is longer than that of the mice injected together with the dendritic cells, it is believed that differentiated mNK cells are more effective for cancer treatment (FIG. 34).

### EXAMPLE 18

### Construction of Human Axl E. coli Expression Vector

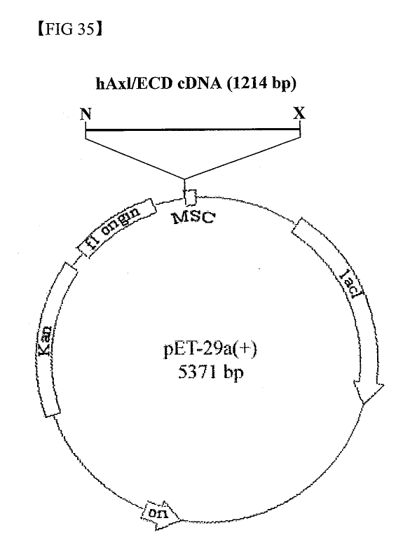

According to the same method as Example 1(1), total RNA was separated from HUVEC(Human Umbilical Vein Endothelial Cell, ATCC) which expresses Axl protein. Using Axl primer (sense; 5′-cca tat gga aag tcc ctt cgt ggg caa c, antisense; 5′-cct cga gca cca gct ggt gga ctg gct g), RT-PCT was carried out to obtain 1214 bp cDNA which corresponds to the extra cellular domain with the signal transfer sequence deleted. It was then cloned into the restriction site between NdeI(1 U, Roche) and XhoI(1 U) of pET29a (50 ng) by using T4 DNA ligase (1 U) so that Axl cDNA(100 ng) is tagged with (his)₆ at its carboxy terminal. Thus obtained clone was named as pET-hAxl/ECD (FIG. 35).

### EXAMPLE 19

### Preparation of Human Axl Antigen

For the expression of Axl-(his)6 protein, E. Coli BL21(DE3) was transformed with pGEX-4T-3-Axl/F and pGEX-4T-3-Axl/R, respectively. Resulting transformed colonies were inoculated into the media (LB broth, Gibco) comprising kanamycin, an antibiotic. 0.5 mM IPTG (Sigma) was further added and the cells were cultured at 25° C. for 4 hrs. Thus obtained floating bacteria cells were centrifuged for 10 min at 6000 rpm. Recovered cells were suspended in 1×PBS. After maintained on ice, the cells were completely broken by sonicator and the supernatant was separated by centrifuge at 12,000 rpm for 30 min. In order to purify the fusion protein from water soluble and insoluble hAxl/ECD present in the supernatant (i.e., bacteria lysate), the lysate was treated with HiTrap™ chelating HP(Amersham pharmacia). Axl-(his)₆ fusion protein was securely obtained.

### EXAMPLE 20

### Preparation of Polyclonal Antibody Specific to Human Axl Protein

After obtaining human recombinant Axl antigen from E. Coli, it was used as an antigen to give anti rabbit human Axl antibody. In order to prepare polyclonal antibodies specific to antigen, 300 μg of human Axl protein solubilized in phosphate buffer in concentration of 1 mg/ml was emulsified with the same amount of complete Freund's adjuvant, and then the resulting mixture was used for the first immunoinjection to a rabbit. After the first injection, the emulsion comprising the antigen in the same amount as the first injection and incomplete Freund's adjuvant was administered to the animal via muscular injection, for the second, the third and the fourth immunoinjection which were carried out with 2 weeks, 1 week and 1 week interval, respectively. Seven days after the fourth injection, sample blood was taken from a hole made into the animal's heart. The blood sample was left at room temperature for 30 min and at 4° C. overnight, thus completely coagulated. Supernatant was obtained by the centrifuge at 2500 rpm for 30 min, which corresponds to blood serum. The resulting serum was diluted five times with 1×PBS and then purified on Protein A column.

### EXAMPLE 21

### Preparation of Monoclonal Antibody Specific to Human Axl protein

Purified human Axl protein was emulsified with the same amount of incomplete Freund's adjuvant, and then the resulting mixture was intraperitoneally injected three times to 6 to 8 week old BALB/c mouse with 2-week interval. After the final injection, formation of the antibody directed against Axl protein was determined by ELISA. After two weeks, the last immunization with 25 μg of human Axl was carried out. Five days later spleen cells were taken from the mouse and mixed with Sp2/0 myeloma cells in 10:1 ratio. Resulting mixture was left inside 50% polyethylene glycol 1500 solution for 3 min to induce cell fusion. After the centrifuge at 1200 rpm for 8 min, cellular precipitate obtained was mixed into HAT RPMI-1640 medium comprising 10% fetal bovine serum so that cell concentration became 3.5×10⁶ cells per one ml of the medium, and this was seeded to 96-well plate (0.1 ml/well) and incubated in 5% CO₂ incubator at 37° C. After three days of the incubation, 0.1 ml of HAT RPMI-1640 medium comprising 10% fetal bovine serum was added into each well and about half of the medium was replaced with the fresh one every 4 days. ELISA was performed for the culture medium and the cells with high titer were recovered from the plate and cultured with the limiting dilution method. ELISA was performed for the culture of 0.5 cell/well/96-well plate and the hybridoma with antigen specificity and high activity was selected. After culture with HAT selection medium, the ability of hybridoma cells for producing the desired antibody was determined by immunoassay. Specifically, human Axl protein used for the immunization above was diluted to 0.1 μg/ml with 0.01M carbonate-bicarbonate buffer (pH 9.6) and the resulting solution was added to the well 50 μL each and coated overnight at 4° C. Then, the wells were washed four times with PBST(phosphate buffer saline, 137 mM NaCl, 2.7 mM KCl, 10 mM Na₂HPO₄, 2 mM KH₂PO₄, 0.15% Tween 20) and blocking was carried out by incubating with 0.1% albumin at 37° C. for 30 min. Culture supernatant was added into the well 50 μL each and the reaction was carried out for 2 hrs at room temperature. Washing with PBST was carried out four times. Anti-mouse immunoglobulin, which is a secondary antibody tagged with biotin, was diluted with 0.1% BSA-PBST to 1 g/ml, and added to the well 50 μL each and reacted for 1 hr at 37° C. Once again, the wells were washed with PBST four times. Streptavidin-Horseradish Peroxidase was diluted 100 times with 0.1% BSA-PBST and introduced to the well 50 μL each and reacted for 30 min at 37° C. The wells were washed with PBST four times again. As a substrate for enzyme reaction, TMB (Tetra-Methylbenzidine) solution was added to the well 50 μL each and reacted at room temperature. After stopping the reaction with 2N sulfuric acid, absorbance was measured at the wavelength of 450 nm using ELISA reader. Cells which reacted positively according to the test for anti-human Axl antibody were cultured by subcloning three times (0.3 cells per well) for monoclonalization, thus obtaining hybridoma which produces anti-human Axl monoclonal antibody. To separate anti-human Axl monoclonal antibody from the culture supernatant of the hybridoma, the antibody was purified by GC column and dialyzed. Anti-human Axl monoclonal antibody was finally obtained.

### EXAMPLE 22

### Construction of Human Gas6 Expression Vector

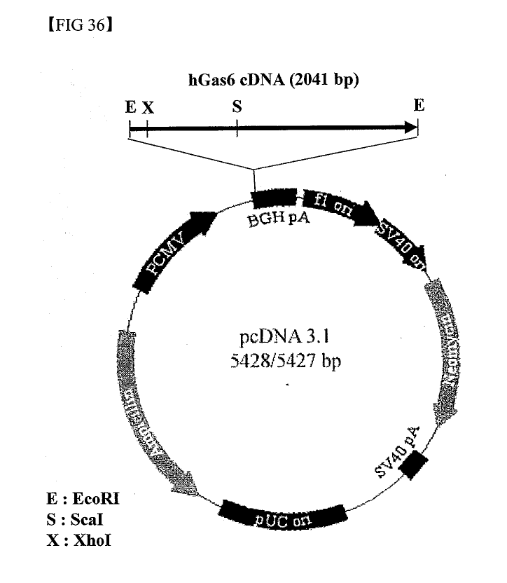

Using the same method as described Example 1(C), total RNA was separated from human cell line HUVEC (American Type Culture collection), which expresses Gas6 protein. Using Gas6 primer (sense; 5′-ggcccgtggccccttcgctct (SEQ ID NO: 24), antisense; ggcctaggctgcggcgggct (SEQ ID NO: 25)), RT-PCR was carried out to amplify 2041 bp cDNA, which was then cloned into PCR coning vector. DNA sequencing was carried out for the clone. Analyzed human Gas6 cDNA was digested with EcoRI(1 U) and purified. Thus obtained human Gas6 cDNA(100 ng) was cloned into pcDNA3.1 vector (50 ng), which has been previously digested with EcoRI(1 U), using T4 DNA ligase (1 U). Consequently pchGas 6 vector which expresses human Gas6 was obtained (FIG. 36).

### EXAMPLE 23

### Preparation of γ-Carboxylated Human Gas6

To prepare γ-carboxylated human Gas6, human 293 cells (ATCC) were transfected with human Gas6 vector pchGas6 using lipfectamine agent (Invitrogen, Carlslbad, Calif.). Resulting transfectants were selected in the medium containing 0.75 mg/ml of G418 antibiotic (Gibco). By using limiting assay, clones which over-express human Gas6 protein were obtained. 293 cells transfected with human Gas6 secretes a great amount of γ-carboxylated human Gas6 protein into culture supernatant. Western blot analysis of such supernatant according the method described in Example 8 confirms that it corresponds to the clone over-expressing human Gas6 protein. Culture supernatant of 293 cells transfected with human Gas6 was used in 1:20 dilution for inducing the differentiation of human NK cells. Activity of γ-carboxylated human Gas6 was confirmed for this culture supernatant.

### EXAMPLE 24

### Differentiation of Human Cord Blood-Derived HSCs Into mNK Cells Using Axl Polyclonal Antibody (Santa Cruz)

(A) Differentiation of Human Cord Blood-Derived HSCs into mNK cells

HSCs were isolated from cord blood in accordance with the follow method. Human cord blood was divided by 25 mL and each contained into 50 mL tube. To this was added 25 mL of 1×PBS and gently mixed. Percoll was aliquotted in 20 mL-volumes into fresh 50-mL tubes. The mixture of 1:1 cord blood and 1×PBS was carefully poured while maintaining the separate layers into the Percoll-containing tube to a final volume of 50 mL. The mixture was then centrifuged under an unfastened break at 2,000 rpm, 25° C. for 20 minutes. After centrifugation, the white-colored layer (cellular layer=about 10 mL) between the yellowish top layer (serum) and transparent bottom layer was collected and transferred into 50 mL tube containing 20 mL of 1×PBS. It was centrifuged at 2,000 rpm, 25° C. for 10 minutes. Once the pellet was submerged at the bottom, the supernatant was discarded. The pellet was taken off by tapping with a hand and was mixed with 10 mL of ACK buffer solution (0.15 M N₄Cl, 1 mM KHCO₃, 0.1 mM EDTA (disodium salt), pH 7.2). The mixture was incubated at 37° C. for 10 minutes. It was centrifuged at 2,500 rpm, 4° C. for 10 minutes. The supernatant was discarded and the pellet was taken off by tapping with a hand. The pellet was suspended into about 2 mL of MACS buffer solution (2 mM EDTA and 0.5% BSA were solved in 1×PBS and filtered) using pipette. The suspension was passed over the filter which had been put on a 50 mL tube. The tube containing cells washed with 10 mL of MACS buffer solution and filtered. This procedure was repeated until the total volume of the resuspended pellet was 50 mL.

Cells contained in 50 mL tube were counted using a hemocytometer (Marienfeld). The cells were centrifuged at 2,000 rpm, 4° C. for 10 minutes and were counted. The two counts were compared and 1×10⁸ was calculated as one reaction. In accordance with the reaction number, the pellet was treated with 1 mL of MACS buffer solution. The solution was contained into 1.5 mL tube which had been prepared according to the reaction number. It was centrifuged at 1,700 rpm for 3 minutes. After the supernatant was discarded, the pellet washed by treating a buffer solution. This washing was repeated three times. Finally, the pellet was treated with 500 μL MACS solution and was mixed with 25 μL of CD34⁺ micro bead and 25 μL of blocking reagent in each 1.5 mL tube. After reaction at 4° C. for 30 minutes, the tube was turned upside down 10 times every 5 minutes. After 30 minutes, the solution was centrifuged at 1,700 rpm for 3 minutes. The supernatant washed out three times. Finally, the pellet was treated with 500 μL of MACS buffer solution. MACS columns were placed in a MACS and were washed with 3 mL of MACS buffer solution. Before the buffer solution was completely emptied from the column, the pellet suspension was carefully poured into the column. Once the pellet suspension was completely poured through the column, 5 mL and 2 mL of MACS buffer solutions were successfully poured through the column to rinse it.

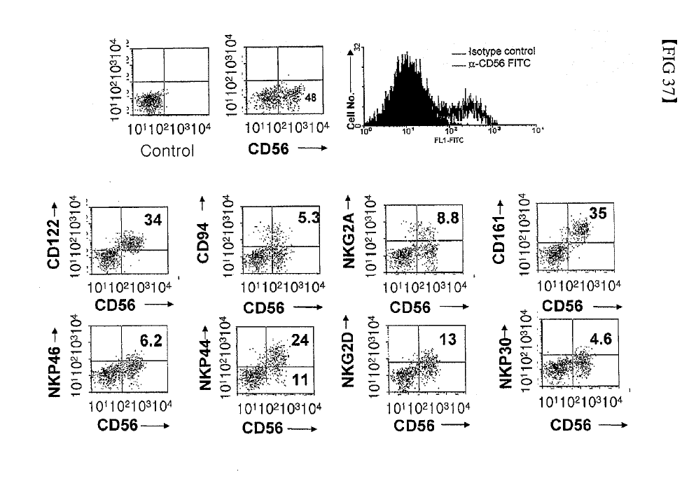

Columns were separated from MACS. 5 mL of MACS buffer solution was added and drained into 15 mL tube. 5 mL of MACS buffer solution was added once more and drained into the tube. The cells were counted and plated at a 24-well plate containing 1 mL of HSC culture solution (human SCF 30 ng/mL (Pepro Tech), human Flt-3L 50 ng/mL (Pepro Tech), human IL-7 10 ng/mL (Pepro Tech)) at 1×10⁶ cells/mL per well. The plate was cultured at 37° C., 5% CO₂ incubator. Isolated CD34+HSCs were suspended in Myelocult H5100 (Gibco) medium containing human SCF (30 ng/mL), Flt-3L (50 ng/mL), and IL-7 (10 ng/mL) at 1×10⁶/mL and were cultured in 24-well plate. The cells were subcultured by replacing half of the medium with a fresh medium every 3 days. These cells were cultured for 14 days to afford human precursor natural killer cells. The resulting pNK cells were co-cultured on MyeloCult medium (Gibco) containing human IL-15 (20 ng/mL, Pepro Tech) along with stromal cells isolated from cord blood (isolation method: CD34⁻ HSCs were isolated from cord blood, plated over 10% RPMI medium and cultured in 5% CO₂ incubator for 2 hours. The cells floated on the culture soup were plated on fresh 10% RPMI medium. After 4 and 7 days, culture solutions were replaced. On day 8, after medium was removed and 3 mL of 1×PBS was added, the culture was stored for 5 minutes in incubator. The culture was treated with 1 mL of trypsin-EDTA for one minute and the cells were treated with 10% RPMI medium. The cells were collected by centrifugation and stromal cells were plated over 24-well plate at 4×10⁴/well.) to afford mature natural killer cells. The purity of the developed mNK cells was analyzed by FACS as described in the above Example 1(B). NKG2A (Pharmingen), CD161 (Pharmingen), NKP46 (Pharmingen), NKP30 (Pharmingen), NKP44 (Pharmingen), NKG2D (Pharmingen), and CD56 (Pharmingen) were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were developed as 8.8%, 35%, 6.2%, 4.6%, 24% and 13%, respectively, as compared to isotype control (FIG. 37).

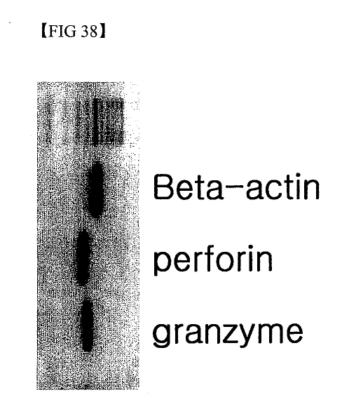

The specific expression of perforin and granzyme genes in the developed mNK cells was ascertained by RT-PCR (FIG. 38). The RT-PCR was performed as described in the above Example 1 (C).

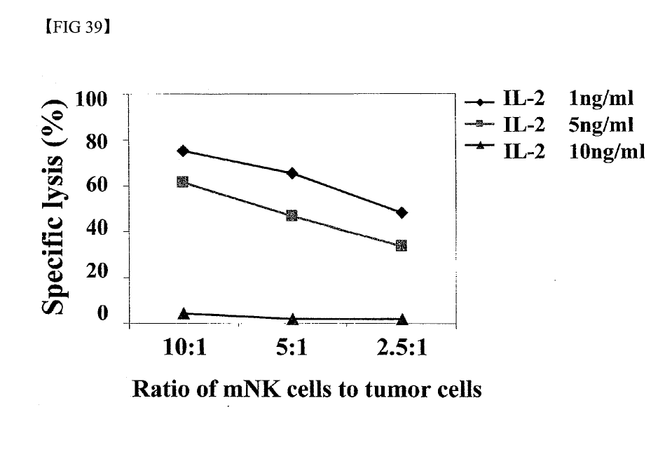

The developed mNK cells were tested for tumor-killing capability. The cells were stimulated with human IL-2 (1, 5 and 10 ng/mL) for 62 hours and were reacted with K562 lymphoma cell (ATCC) labeled with ⁵¹Cr (50 uci) for 2 hours at ratio of 10:1, 5:1, and 2.5:1. After 4 hours, the amount of ⁵¹Cr secreted from culture supernatant was measured. As results, tumor-killing capabilities was augmented by increasing the number of mNK cells (FIG. 39).

(B) Differentiation of Human pNK Cells into mNK Cells without Stromal Cells

In accordance with the same manner as described in the above (A), human pNK cells were differentiated into mNK cells in the absence of human stromal cells. The expression of mNK-associated surface molecules including CD56 (Pharmingen) was analyzed by FACS in accordance with the same method as described in the above Example 1(B). As results, the expression degree in the absence of stromal cells was considerably lower than that under co-culturing with stromal cells. This proves that it is preferred to use stromal cells in the differentiation of pNK cells into mNK cells.

(C) Differentiation of Human Cord Blood-Derived HSCs into mNK Cells using Axl Polyclonal Antibody (Santa Cruz)

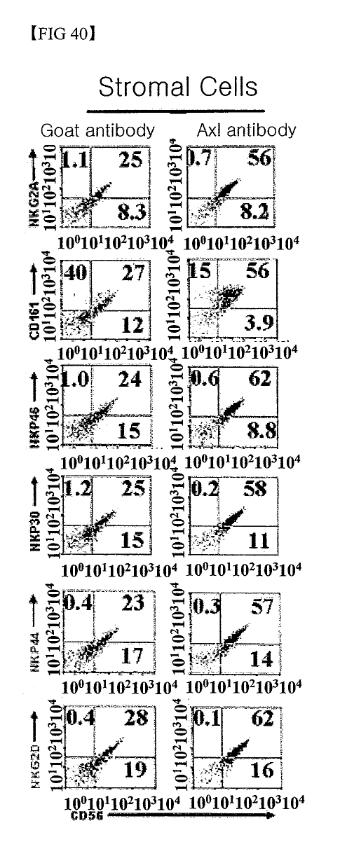

The pNK cells obtained in the above (A) were differentiated in accordance with the same manner as described in the above (A) but using 1 μg/mL of Axl polyclonal antibody (Santa Cruz) for 14 days instead of IL-15. The development degree of human mNK cells was ascertained by FACS in accordance with the same method as described in the above Example 1(B). NKG2A (Pharmingen), CD161 (Pharmingen), NKP46 (Pharmingen), NKP30 (Pharmingen), NKP44 (Pharmingen), NKG2D (Pharmingen), and CD56 (Pharmingen) were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using Axl polyclonal antibody than by using goat antibody (control) (FIG. 40).

In the same manner as described above, pNK cells were treated with the 1:1 combination (1 μg/mL) of Axl polyclonal antibody (Santa Cruz) and IL-15 (Pepro Tech) under the same conditions and the purity was ascertained. As results, the development degree of mNK cells was slightly increased by the combination, in comparison with by Axl polyclonal antibody alone.

### EXAMPLE 25

### Differentiation of Human Cord Blood-Derived HSCs into mNK Cells using Human Axl Polyclonal Antibody

The pNK cells obtained in the above Example 24(A) were differentiated in accordance with the same manner as described in the above Example 24(A) but using 1 μg/mL of human Axl polyclonal antibody obtained in the above Example 20 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56+NKP30⁺, CD56⁺NKP44⁺ and CD56⁺NKG2D⁺ cells were about two to three times more developed by using human Axl polyclonal antibody than by using goat antibody (control). This result is similar to that differentiated into mNK cells by using commercially available Axl polyclonal antibody.

In the same manner as described above, pNK cells were treated with the 1:1 combination (1 μg/mL) of human Axl polyclonal antibody and IL-15 (Pepro Tech) under the same conditions and the purity was ascertained. As results, the development degree of mNK cells was slightly increased by the combination, in comparison with by human Axl polyclonal antibody alone.

### EXAMPLE 26

### Differentiation of Human Cord Blood-Derived HSCs Into mNK Cells using Human Axl Monoclonal Antibody

The pNK cells obtained in the above Example 24(A) were differentiated in accordance with the same manner as described in the above Example 24(A) but using 1 μg/mL of human Axl monoclonal antibody obtained in the above Example 21 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using human Axl monoclonal antibody than by using goat antibody (control). This result is similar to that differentiated into mNK cells by using commercially available Axl polyclonal antibody.

In the same manner as described above, pNK cells were treated with the 1:1 combination (1 μg/mL) of human Axl monoclonal antibody and IL-15 (Pepro Tech) under the same conditions and the purity was ascertained. As results, the development degree of mNK cells was slightly increased by the combination, in comparison with by human Axl monoclonal antibody alone.

### EXAMPLE 27

### Differentiation of Human Cord Blood-Derived HSCs into mNK Cells using γ-Carboxylated Human Gas6

The pNK cells obtained in the above Example 24(A) were differentiated in accordance with the same manner as described in the above Example 24(A) but using 1 μg/mL of 1:20 diluted culture supernatant of Gas6-transfectant obtained in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using γ-carboxylated human Gas6 than by using culture supernatant of vector-transfected cell (control). This result is similar to that differentiated into mNK cells by using human Axl polyclonal antibody or Axl monoclonal antibody.

In the same manner as described above, pNK cells were treated with the 1:1 combination (1 μg/mL) of γ-carboxylated human Gas6 and IL-15 (Pepro Tech) under the same conditions and the purity was ascertained. As results, the development degree of mNK cells was slightly increased by the combination, in comparison with by γ-carboxylated human Gas6 alone.

### EXAMPLE 28

### Differentiation of Human Cord Blood-Derived HSCs into mNK Cells using Axl Polyclonal Antibody (Santa Cruz) and γ-Carboxylated Human Gas6

The pNK cells obtained in the above Example 24(A) were differentiated in accordance with the same manner as described in the above Example 24(A) but using 1 μg/mL of Axl polyclonal antibody (Santa Cruz) and 1:20 diluted culture supernatant of Gas6-transfectant obtained in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were more slightly developed by using the combination of γ-carboxylated human Gas6 and Axl polyclonal antibody than by using γ-carboxylated human Gas6 alone or Axl polyclonal antibody alone (control).

In the same manner as described above, pNK cells were treated with the combination of Axl polyclonal antibody and γ-carboxylated human Gas6 along with IL-15 (1:1 ratio with antibody) under the same conditions and the purity was ascertained. As results, the development degree of mNK cells was slightly increased by the combination of the three ingredients, in comparison with by the combination of Axl polyclonal antibody and γ-carboxylated human Gas6.

### EXAMPLE 29

### Differentiation of Human Cord Blood-Derived HSCs into mNK Cells using Human Axl Polyclonal Antibody and γ-Carboxylated Human Gas6

The pNK cells obtained in the above Example 24(A) were differentiated in accordance with the same manner as described in the above Example 24(A) but using human Axl polyclonal antibody obtained in the above Example 20 and γ-carboxylated human Gas6 obtained in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were more slightly developed by using the combination of γ-carboxylated human Gas6 and human Axl polyclonal antibody than by using γ-carboxylated human Gas6 alone or human Axl polyclonal antibody alone (control). This result is similar to that differentiated into mNK cells by using the combination of commercially available Axl polyclonal antibody and γ-carboxylated human Gas6.

In the same manner as described above, pNK cells were treated with the combination of Axl polyclonal antibody and γ-carboxylated human Gas6 along with IL-15 (1:1 ratio with antibody) under the same conditions and the purity was ascertained. As results, the development degree of mNK cells was slightly increased by the combination of the three ingredients, in comparison with by the combination of human Axl polyclonal antibody and γ-carboxylated human Gas6.

### EXAMPLE 30

### Differentiation of Human Cord Blood-Derived HSCs into mNK Cells using Human Axl Monoclonal Antibody and γ-Carboxylated Human Gas6

The pNK cells obtained in the above Example 24(A) were differentiated in accordance with the same manner as described in the above Example 24(A) but using human Axl monoclonal antibody obtained in the above Example 21 and γ-carboxylated human Gas6 obtained in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were more slightly developed by using the combination of γ-carboxylated human Gas6 and human Axl monoclonal antibody than by using γ-carboxylated human Gas6 alone or human Axl monoclonal antibody alone (control). This result is similar to that differentiated into mNK cells by using the combination of commercially available Axl polyclonal antibody and γ-carboxylated Gas6.

In the same manner as described above, pNK cells were treated with the combination of Axl monoclonal antibody and γ-carboxylated human Gas6 along with IL-15 (1:1 ratio with antibody) under the same conditions and the purity was ascertained. As results, the development degree of mNK cells was slightly increased by the combination of the three ingredients, in comparison with by the combination of human Axl monoclonal antibody and γ-carboxylated human Gas6.

### EXAMPLE 31

### Anti-Tumor Activity in an Animal Tumor Model of mNK Cells Developed from Human Cord Blood-Derived HSCs by Axl Polyclonal Antibody (Santa Cruz)

Five-week-old male nude Balb/c mice (immunodeficient transgenic mice purchased from Orient Bio) were used to see whether human mNK cells developed by Axl polyclonal antibody (Santa Cruz) exert tumor-killing activity. The nude mice were housed in a germ-free animal room according to an animal management guideline. Human tumor cell lines (for example, gastric carcinoma KCLB cat. no. 00638, uterine carcinoma KCLB cat. no. 10002, breast carcinoma KCLB cat. no. 30022, renal carcinoma KCLB cat. no. 30044, melanoma KCLB cat. no. 30068, lung carcinoma KCLB cat. no. 30053, ovarian carcinoma KCLB cat. no. 30077, etc.) were cultured on RPMI 1640 media (Gibco) containing 10% fetal bovine serum at 37° C. in CO₂ incubator. The cultured tumor cell lines were washed with 1×PBS and were treated with trypsin-EDTA (Gibco). The trpsin-EDTA was removed from cell lines which was then suspended in 1×PBS. The cells were counted to prepare 10⁸ cells/0.1 μL. Nude mice were subcutaneously injected with 10⁸ tumor cells by an insulin syringe (Pharmingen) and were grown for about one week until they were about 0.8 mm in diameter. Human mNK cells (1×10⁶) developed by Axl polyclonal antibody (Santa Cruz) were activated with human IL-2 (10 ng/mL). The formed tumor tissue site was injected with the activated mNK cells. Tumor volume was measured every hour. As results, tumor volume of the test group injected with human mNK cells developed by Axl polyclonal antibody (Santa Cruz) was remarkably reduced in comparison with that of the control group injected with human mNK cells developed by goat antibody.

### EXAMPLE 32

### Anti-Tumor Activity in an Animal Tumor Model of mNK Cells Developed from Human Cord Blood-Derived HSCs by Human Axl Polyclonal Antibody

The same experimental method as described in the above Example 31 was conducted to see whether human mNK cells developed by human Axl polyclonal antibody exert tumor-killing activity. As results, tumor volume of the test group injected with human mNK cells developed by human Axl polyclonal antibody was remarkably reduced in comparison with that of the control group injected with human mNK cells developed by goat antibody. This is similar with the result obtained by using mNK cells developed by commercially available Axl polyclonal antibody.

### EXAMPLE 33

### Anti-Tumor Activity in an Animal Tumor Model of mNK Cells Developed from Human Cord Blood-Derived HSCs by Human Axl Monoclonal Antibody

The same experimental method as described in the above Example 31 was conducted to see whether human mNK cells developed by human Axl monoclonal antibody exert tumor-killing activity. As results, tumor volume of the test group injected with human mNK cells developed by human Axl monoclonal antibody was remarkably reduced in comparison with that of the control group injected with human mNK cells developed by goat antibody. This is similar with the result obtained by using mNK cells developed by commercially available Axl polyclonal antibody.

### EXAMPLE 34

### Anti-Tumor Activity in an Animal Tumor Model of mNK Cells Developed from Human Cord Blood-Derived HSCs by γ-Carboxylated Human Gas6

The same experimental method as described in the above Example 31 was conducted to see whether human mNK cells developed by γ-carboxylated human Gas6 exert tumor-killing activity. As results, tumor volume of the test group injected with human mNK cells developed by γ-carboxylated human Gas6 was remarkably reduced in comparison with that of the control group injected with human mNK cells developed by culture supernatant of vector-containing transfectant. This is similar with the result obtained by using mNK cells developed by commercially available Axl polyclonal antibody.

### EXAMPLE 35

### Anti-Tumor Activity in an Animal Tumor Model of mNK Cells Developed from Human Cord Blood-Derived HSCs by the Combination of Axl Polyclonal Antibody (Santa Cruz) and γ-Carboxylated Human Gas6

The same experimental method as described in the above Example 31 was conducted to see whether human mNK cells developed by the combination of Axl polyclonal antibody (Santa Cruz) and γ-carboxylated human Gas6 exert tumor-killing activity. As results, tumor volume of the test group injected with human mNK cells developed by the combination of Axl polyclonal antibody (Santa Cruz) and γ-carboxylated human Gas6 was more slightly reduced in comparison with that of the control group injected with human mNK cells developed by Axl polyclonal antibody (Santa Cruz) or the control group injected with human mNK cells developed by culture supernatant of vector-containing transfectant.

### EXAMPLE 36

### Anti-Tumor Activity in an Animal Tumor Model of mNK Cells Developed from Human Cord Blood-Derived HSCs by the Combination of Human Axl Polyclonal Antibody and γ-Carboxylated Human Gas6

The same experimental method as described in the above Example 31 was conducted to see whether human mNK cells developed by the combination of human Axl polyclonal antibody and γ-carboxylated human Gas6 exert tumor-killing activity. As results, tumor volume of the test group injected with human mNK cells developed by the combination of human Axl polyclonal antibody and γ-carboxylated human Gas6 was more slightly reduced in comparison with that of the control group injected with human mNK cells developed by human Axl polyclonal antibody or the control group injected with human mNK cells developed by culture supernatant of vector-containing transfectant. This is similar with the result obtained by using mNK cells developed by the combination of commercially available Axl polyclonal antibody and culture supernatant of γ-carboxylated human Gas6-containing transfectant.

### EXAMPLE 37

### Anti-Tumor Activity in an Animal Tumor Model of mNK Cells Developed from Human Cord Blood-Derived HSCs by the Combination of Human Axl Monoclonal Antibody and γ-Carboxylated Human Gas6

The same experimental method as described in the above Example 31 was conducted to see whether human mNK cells developed by the combination of human Axl monoclonal antibody and γ-carboxylated human Gas6 exert tumor-killing activity. As results, tumor volume of the test group injected with human mNK cells developed by the combination of human Axl monoclonal antibody and γ-carboxylated human Gas6 was more slightly reduced in comparison with that of the control group injected with human mNK cells developed by human Axl monoclonal antibody or the control group injected with human mNK cells developed by culture supernatant of vector-containing transfectant. This is similar with the result obtained by using mNK cells developed by the combination of commercially available Axl polyclonal antibody and culture supernatant of γ-carboxylated human Gas6-containing transfectant.

### EXAMPLE 38

### Differentiation of Human Bone Marrow-Derived HSCs into mNK Cells using Axl Polyclonal Antibody (Santa Cruz)

(A) Differentiation of Human Bone Marrow-Derived HSCs into mNK cells

Human bone marrow was divided by 25 mL which was then contained into 50 mL tube. In accordance with the same method as described in the above Example 24(A), HSCs were differentiated via pNK cells into mNK cells. Human stromal cells used in the procedure were isolated from human bone marrow in accordance with the same method as described in the above Example 24(A) for the isolation of stromal cells from cord blood.

(B) Development of mNK cells using Axl polyclonal antibody (Santa Cruz)

The pNK cells obtained in the above (A) were differentiated in accordance with the same manner as described in the above (A) but using 1 μg/mL of Axl polyclonal antibody (Santa Cruz) for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using Axl polyclonal antibody than by using goat antibody (control).

In the same manner as described above, pNK cells were treated with the 1:1 combination (1 μg/mL) of Axl polyclonal antibody (Santa Cruz) and IL-15 (Pepro Tech) under the same conditions and the purity was ascertained. As results, the development degree of mNK cells was slightly increased by the combination, in comparison with by Axl polyclonal antibody alone.

### EXAMPLE 39

### Differentiation of Human Bone Marrow-Derived HSCs into mNK Cells using Human Axl Polyclonal Antibody

The pNK cells obtained in the above Example 38(A) were differentiated in accordance with the same manner as described in the above Example 38(A) but using 1 μg/mL of human Axl polyclonal antibody for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using human Axl polyclonal antibody than by using goat antibody (control). This result is similar to that differentiated into mNK cells by using commercially available Axl polyclonal antibody.

In the same manner as described above, pNK cells were treated with the 1:1 combination (1 μg/mL) of human Axl polyclonal antibody and IL-15 (Pepro Tech) under the same conditions and the purity was ascertained. As results, the development degree of mNK cells was slightly increased by the combination, in comparison with by Axl polyclonal antibody alone.

### EXAMPLE 40

### Differentiation of Human Bone Marrow-Derived HSCs into mNK Cells using Human Axl Monoclonal Antibody

The pNK cells obtained in the above Example 38(A) were differentiated in accordance with the same manner as described in the above Example 38(A) but using 1 μg/mL of human Axl monoclonal antibody for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using human Axl monoclonal antibody than by using goat antibody (control). This result is similar to that differentiated into mNK cells by using commercially available Axl polyclonal antibody.

In the same manner as described above, pNK cells were treated with the 1:1 combination (1 μg/mL) of human Axl monoclonal antibody and IL-15 (Pepro Tech) under the same conditions and the purity was ascertained. As results, the development degree of mNK cells was slightly increased by the combination, in comparison with by Axl monoclonal antibody alone.

### EXAMPLE 41

### Differentiation of Human Bone Marrow-Derived HSCs into mNK Cells using γ-Carboxylated Human Gas6

The pNK cells obtained in the above Example 38(A) were differentiated in accordance with the same manner as described in the above Example 38(A) but using 1:20 diluted culture supernatant of Gas6-containing transfectant produced in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were about two to three times more developed by using γ-carboxylated human Gas6 than by using culture supernatant of vector-containing transfectant (control). This result is similar to that differentiated into mNK cells by using human Axl polyclonal antibody or Axl monoclonal antibody.

In the same manner as described above, pNK cells were treated with the 1:1 combination (1 μg/mL) of γ-carboxylated human Axl and IL-15 (Pepro Tech) under the same conditions and the purity was ascertained. As results, the development degree of mNK cells was slightly increased by the combination, in comparison with by γ-carboxylated human Gas6 alone.

### EXAMPLE 42

### Differentiation of Human Bone Marrow-Derived HSCs into mNK Cells using the Combination of Axl Polyclonal Antibody (Santa Cruz) and γ-Carboxylated Human Gas6

The pNK cells obtained in the above Example 38(A) were differentiated in accordance with the same manner as described in the above Example 38(A) but using the combination of Axl polyclonal antibody (Santa Cruz) and 1:20 diluted culture supernatant of Gas6-containing transfectant produced in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were slightly more developed by using the combination of Axl polyclonal antibody (Santa Cruz) and 1:20 diluted culture supernatant of Gas6-containing transfectant than by using Axl polyclonal antibody (Santa Cruz) alone (control) or 1:20 diluted culture supernatant of Gas6-containing transfectant alone (control).

In the same manner as described above, pNK cells were treated with the combination of Axl polyclonal antibody (Santa Cruz) and 1:20 diluted culture supernatant of Gas6-containing transfectant along with IL-15 (Pepro Tech) (1:1 ratio of antibody) under the same conditions and the purity was ascertained. As results, the development degree of mNK cells was slightly increased by the combination of the above three ingredients, in comparison with by the combination of Axl polyclonal antibody and γ-carboxylated human Gas6.

### EXAMPLE 43

### Differentiation of Human Bone Marrow-Derived HSCs into mNK Cells using the Combination of Human Axl Polyclonal Antibody and γ-Carboxylated human Gas6

The pNK cells obtained in the above Example 38(A) were differentiated in accordance with the same manner as described in the above Example 38(A) but using the combination of 1 μg/mL of human Axl polyclonal antibody produced in the above Example 19 and 1:20 diluted culture supernatant of Gas6-containing transfectant produced in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were slightly more developed by using the combination of human Axl polyclonal antibody and 1:20 diluted culture supernatant of Gas6-containing transfectant than by using human Axl polyclonal antibody alone (control) or 1:20 diluted culture supernatant of Gas6-containing transfectant alone (control). This result is similar to that differentiated into mNK cells by using the combination of commercially available Axl polyclonal antibody and culture supernatant of Gas6-containing transfectant.

In the same manner as described above, pNK cells were treated with the combination of human Axl polyclonal antibody and γ-carboxylated human Gas6 along with IL-15 (Pepro Tech) (1:1 ratio of the antibody) under the same conditions and the purity was ascertained. As results, the development degree of mNK cells was slightly increased by the combination of the above three ingredients, in comparison with by the combination of human Axl polyclonal antibody and γ-carboxylated human Gas6.

### EXAMPLE 44

### Differentiation of Human Bone Marrow-Derived HSCs into mNK Cells using the Combination of Human Axl Monoclonal Antibody and γ-Carboxylated Human Gas6

The pNK cells obtained in the above Example 38(A) were differentiated in accordance with the same manner as described in the above Example 38(A) but using the combination of 1 μg/mL of human Axl monoclonal antibody produced in the above Example 21 and 1:20 diluted culture supernatant of Gas6-containing transfectant produced in the above Example 23 for 14 days instead of IL-15. The development degree of human mNK cells was ascertained by FACS in accordance with the same method as described in the above Example 1(B). NKG2A, CD161, NKP46, NKP30, NKP44, NKG2D, and CD56 were used as markers for human mNK cells. As results, CD56⁺NKG2A⁺, CD56⁺CD161⁺, CD56⁺NKP46⁺, CD56⁺NKP30⁺, CD56⁺NKP44⁺ and CD56⁺NKG2D⁺ cells were slightly more developed by using the combination of human Axl monoclonal antibody and culture supernatant of Gas6-containing transfectant than by using human Axl monoclonal antibody alone (control) or culture supernatant of Gas6-containing transfectant alone (control). This result is similar to that differentiated into mNK cells by using the combination of commercially available Axl polyclonal antibody and culture supernatant of Gas6-containing transfectant.

In the same manner as described above, pNK cells were treated with the combination of Axl monoclonal antibody and γ-carboxylated human Gas6 along with IL-15 (Pepro Tech) (1:1 ratio of the antibody) under the same conditions and the purity was ascertained. As results, the development degree of mNK cells was slightly increased by the combination of the above three ingredients, in comparison with by the combination of human Axl monoclonal antibody and γ-carboxylated human Gas6.

### EXAMPLE 45

### Anti-Tumor Activity in an Animal Tumor Model of mNK Cells Developed from Human Bone Marrow-Derived HSCs by Axl Polyclonal Antibody (Santa Cruz)

The same experimental method as described in the above Example 31 was conducted to see whether human mNK cells developed by Axl polyclonal antibody (Santa Cruz) exert tumor-killing activity. As results, tumor volume of the test group injected with human mNK cells developed by Axl polyclonal antibody (Santa Cruz) was remarkably reduced in comparison with that of the control group injected with human mNK cells developed by goat antibody.

### EXAMPLE 46

### Anti-Tumor Activity in an Animal Tumor Model of mNK Cells Developed from Human Bone Marrow-Derived HSCs by Human Axl Polyclonal Antibody

The same experimental method as described in the above Example 31 was conducted to see whether human mNK cells developed by human Axl polyclonal antibody exert tumor-killing activity. As results, tumor volume of the test group injected with human mNK cells developed by human Axl polyclonal antibody was remarkably reduced in comparison with that of the control group injected with human mNK cells developed by goat antibody. This is similar with the result obtained by using mNK cells developed by commercially available Axl polyclonal antibody.

### EXAMPLE 47

### Anti-Tumor Activity in an Animal Tumor Model of mNK Cells Developed from Human Bone Marrow-Derived HSCs by Human Axl Monoclonal Antibody

The same experimental method as described in the above Example 31 was conducted to see whether human mNK cells developed by human Axl monoclonal antibody exert tumor-killing activity. As results, tumor volume of the test group injected with human mNK cells developed by human Axl monoclonal antibody was remarkably reduced in comparison with that of the control group injected with human mNK cells developed by goat antibody. This is similar with the result obtained by using mNK cells developed by commercially available Axl polyclonal antibody.

### EXAMPLE 48

### Anti-Tumor Activity in an Animal Tumor Model of mNK Cells Developed from Human Bone Marrow-Derived HSCs by γ-Carboxylated Human Gas6

The same experimental method as described in the above Example 31 was conducted to see whether human mNK cells developed by γ-carboxylated human Gas6 exert tumor-killing activity. As results, tumor volume of the test group injected with human mNK cells developed by γ-carboxylated human Gas6 was remarkably reduced in comparison with that of the control group injected with human mNK cells developed by culture supernatant of vector-containing transfectant. This is similar with the result obtained by using mNK cells developed by commercially available Axl polyclonal antibody.

### EXAMPLE 49

### Anti-Tumor Activity in an Animal Tumor Model of mNK Cells Developed from Human Bone Marrow-Derived HSCs by the Combination of Axl Polyclonal Antibody (Santa Cruz) and γ-Carboxylated Human Gas6

The same experimental method as described in the above Example 31 was conducted to see whether human mNK cells developed by the combination of Axl polyclonal antibody (Santa Cruz) and γ-carboxylated human Gas6 exert tumor-killing activity. As results, tumor volume of the test group injected with human mNK cells developed by the combination of Axl polyclonal antibody (Santa Cruz) and γ-carboxylated human Gas6 was more slightly reduced in comparison with that of the control group injected with human mNK cells developed by Axl polyclonal antibody (Santa Cruz) or the control group injected with human mNK cells developed by culture supernatant of vector-containing transfectant.

### EXAMPLE 50

### Anti-Tumor Activity in an Animal Tumor Model of mNK Cells Developed from Human Bone Marrow-Derived HSCs by the Combination of Human Axl Polyclonal Antibody and γ-Carboxylated Human Gas6

The same experimental method as described in the above Example 31 was conducted to see whether human mNK cells developed by the combination of human Axl polyclonal antibody and γ-carboxylated human Gas6 exert tumor-killing activity. As results, tumor volume of the test group injected with human mNK cells developed by the combination of human Axl polyclonal antibody and γ-carboxylated human Gas6 was more slightly reduced in comparison with that of the control group injected with human mNK cells developed by human Axl polyclonal antibody or the control group injected with human mNK cells developed by culture supernatant of vector-containing transfectant. This is similar with the result obtained by using mNK cells developed by the combination of commercially available Axl polyclonal antibody and culture supernatant of γ-carboxylated human Gas6-containing transfectant.

### EXAMPLE 51

### Anti-Tumor Activity in an Animal Tumor Model of mNK Cells Developed from Human Bone Marrow-Derived HSCs by the Combination of Human Axl Monoclonal Antibody and γ-Carboxylated Human Gas6

The same experimental method as described in the above Example 31 was conducted to see whether human mNK cells developed by the combination of human Axl monoclonal antibody and γ-carboxylated human Gas6 exert tumor-killing activity. As results, tumor volume of the test group injected with human mNK cells developed by the combination of human Axl monoclonal antibody and γ-carboxylated human Gas6 was more slightly reduced in comparison with that of the control group injected with human mNK cells developed by human Axl monoclonal antibody or the control group injected with human mNK cells developed by culture supernatant of vector-containing transfectant. This is similar with the result obtained by using mNK cells developed by the combination of commercially available Axl polyclonal antibody and culture supernatant of γ-carboxylated human Gas6-containing transfectant.

### EXAMPLE 52

### Differentiation of Human Peripheral Blood-Derived HSCs into mNK Cells using Axl Polyclonal Antibody (Santa Cruz)

(A) Differentiation of Human Peripheral Blood-Derived HSCs into mNK Cells

Human peripheral blood was divided by 25 mL which was then contained into 50 mL tube. In accordance with the same method as described in the above Example 24(A), HSCs were differentiated via pNK cells into mNK cells. Human stromal cells used in the procedure were isolated from human peripheral blood in accordance with the same method as described in the above Example 24(A) for the isolation of stromal cells from cord blood.

(B) Development of mNK Cells using Axl Polyclonal Antibody (Santa Cruz)

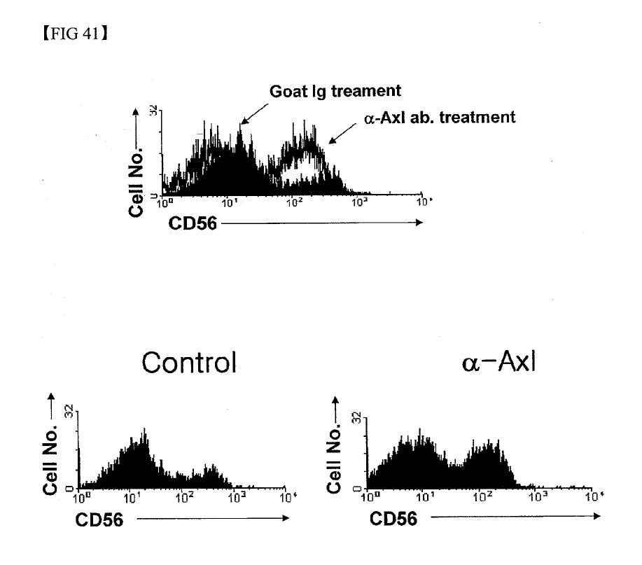

The pNK cells obtained in the above (A) were differentiated in accordance with the same manner as described in the above (A) but using 1 μg/mL of Axl polyclonal antibody (Santa Cruz) for 14 days instead of IL-15. The development degree of human mNK cells was ascertained by FACS in accordance with the same method as described in the above Example 1(B). CD56 were used as markers for human mNK cells. As results, CD56+ cells were about two to three times more developed by using Axl polyclonal antibody than by using goat antibody (control) (FIG. 41).

In the same manner as described above, pNK cells were treated with the 1:1 combination (1 μg/mL) of Axl polyclonal antibody (Santa Cruz) and IL-15 (Pepro Tech) under the same conditions and the purity was ascertained. As results, the development degree of mNK cells was slightly increased by the combination, in comparison with by Axl polyclonal antibody alone.

### EXAMPLE 53

### Treatment of Cancer in Patients with mNK Cells Differentiated from Patient Peripheral Blood Stem Cells using Axl Antibody (Santa Cruz)

Two colon cancer patients with distant metastasis, one breast cancer patient recurred with lung metastasis, one non-Hodgkin's lymphoma patient, and one acute lymphocytic leukemia patient were volunteered to take part in this immunotherapy. The patients received G-CSF starting 1 to 5 days after completion of chemotherapy according to the individual treatment protocol in dosages of 600-900 μg/d s.c. until the end of the collection period. CD34⁺ PBSCs were monitored daily as soon as the WBC recovered (>1×10⁹/1 PB). Blood sample was taken from the patients when there were 20×10⁶ CD34⁺ cells/1 PB. The absolute number of CD34⁺ cells was evaluated by flow cytometry using a FACScan analyzer (Becton Dickinson/Aria) and appropriate isotype-matched, negative control. As described in Example 52, the hematopoietic cells were separated from human peripheral blood and differentiated into mature natural killer cells, which were then activated with 10 ng/ml of IL-2. The resulting mNK cells were injected to the patient for autoimmune therapy. Substantial decrease in tumor size was observed for all patients.

### EXAMPLE 54

### Treatment of Cancer in Patients with mNK Cells Differentiated from Patient Peripheral Blood Stem Cells using Human Axl Polyclonal Antibody

Cancer patients exhibiting similar patient profiles to those in Example 53 volunteer to take part in the experimental immunotherapy. The patients receive G-CSF starting 1 to 5 days after completion of chemotherapy according to the individual treatment protocol in dosages of 600-900 μg/d s.c. until the end of the collection period. CD34+PBSCs are monitored daily as soon as the WBC recovers (>1×10⁹/1 PB). Blood samples are obtained from the patients when there are 20×10⁶ CD34⁺ cells/1 PB. The absolute number of CD34⁺ cells is evaluated by flow cytometry using a FACScan analyzer (Becton Dickinson/Aria) and appropriate isotype-matched, negative control. The hematopoietic cells are separated from human peripheral blood according to the method described in Example 52 and differentiated into mature natural killer cells according to the method described in the Example 24(A) as modified by the procedure of Example 25. The mature natural killer cells are then activated with 10 ng/ml of IL-2. The resulting mNK cells are injected to the patient for autoimmune therapy. Substantial decrease in tumor size is observed for all patients.

### EXAMPLE 55

### Treatment of Cancer in Patients with mNK Cells Differentiated from Patient Peripheral Blood Stem Cells using Human Axl Monoclonal Antibody

Cancer patients exhibiting similar patient profiles to those in Example 53 volunteer to take part in the experimental immunotherapy. The patients receive G-CSF starting 1 to 5 days after completion of chemotherapy according to the individual treatment protocol in dosages of 600-900 μg/d s.c. until the end of the collection period. CD34+PBSCs are monitored daily as soon as the WBC recovers (>1×10⁹/1 PB). Blood samples are obtained from the patients when there are 20×10⁶ CD34⁺ cells/1 PB. The absolute number of CD34⁺ cells is evaluated by flow cytometry using a FACScan analyzer (Becton Dickinson/Aria) and appropriate isotype-matched, negative control. The hematopoietic cells are separated from human peripheral blood according to the method described in Example 52 and differentiated into mature natural killer cells according to the method described in the Example 24(A) as modified by the procedure of Example 26. The mature natural killer cells are then activated with 10 ng/ml of IL-2. The resulting mNK cells are injected to the patient for autoimmune therapy. Substantial decrease in tumor size is observed for all patients.

### EXAMPLE 56

### Treatment of Cancer in Patients with mNK Cells Differentiated from Patient Peripheral Blood Stem Cells using γ-Carboxylated Human Gas6

Cancer patients exhibiting similar patient profiles to those in Example 53 volunteer to take part in the experimental immunotherapy. The patients receive G-CSF starting 1 to 5 days after completion of chemotherapy according to the individual treatment protocol in dosages of 600-900 μg/d s.c. until the end of the collection period. CD34+PBSCs are monitored daily as soon as the WBC recovers (>1×10⁹/1 PB). Blood samples are obtained from the patients when there are 20×10⁶ CD34⁺ cells/1 PB. The absolute number of CD34⁺ cells is evaluated by flow cytometry using a FACScan analyzer (Becton Dickinson/Aria) and appropriate isotype-matched, negative control. The hematopoietic cells are separated from human peripheral blood according to the method described in Example 52 and differentiated into mature natural killer cells according to the method described in the Example 24(A) as modified by the procedure of Example 27. The mature natural killer cells are then activated with 10 ng/ml of IL-2. The resulting mNK cells are injected to the patient for autoimmune therapy. Substantial decrease in tumor size is observed for all patients.

### EXAMPLE 57

### Treatment of Cancer in Patients with mNK Cells Differentiated from Patient Peripheral Blood Stem Cells using Human Axl Polyclonal Antibody and γ-Carboxylated Human Gas6

Cancer patients exhibiting similar patient profiles to those in Example 53 volunteer to take part in the experimental immunotherapy. The patients receive G-CSF starting 1 to 5 days after completion of chemotherapy according to the individual treatment protocol in dosages of 600-900 μg/d s.c. until the end of the collection period. CD34+PBSCs are monitored daily as soon as the WBC recovers (>1×10⁹/1 PB). Blood samples are obtained from the patients when there are 20×10⁶ CD34⁺ cells/1 PB. The absolute number of CD34⁺ cells is evaluated by flow cytometry using a FACScan analyzer (Becton Dickinson/Aria) and appropriate isotype-matched, negative control. The hematopoietic cells are separated from human peripheral blood according to the method described in Example 52 and differentiated into mature natural killer cells according to the method described in the Example 24(A) as modified by the procedure of Example 29. The mature natural killer cells are then activated with 10 ng/ml of IL-2. The resulting mNK cells are injected to the patient for autoimmune therapy. Substantial decrease in tumor size is observed for all patients.

### EXAMPLE 58

### Treatment of Cancer in Patients with mNK Cells Differentiated from Patient Peripheral Blood Stem Cells using Human Axl Monoclonal Antibody and γ-Carboxylated Human Gas6

Cancer patients exhibiting similar patient profiles to those in Example 53 volunteer to take part in the experimental immunotherapy. The patients receive G-CSF starting 1 to 5 days after completion of chemotherapy according to the individual treatment protocol in dosages of 600-900 μg/d s.c. until the end of the collection period. CD34⁺ PBSCs are monitored daily as soon as the WBC recovers (>1×10⁹/1 PB). Blood samples are obtained from the patients when there are 20×10⁶ CD34⁺ cells/1 PB. The absolute number of CD34⁺ cells is evaluated by flow cytometry using a FACScan analyzer (Becton Dickinson/Aria) and appropriate isotype-matched, negative control. The hematopoietic cells are separated from human peripheral blood according to the method described in Example 52 and differentiated into mature natural killer cells according to the method described in the Example 24(A) as modified by the procedure of Example 30. The mature natural killer cells are then activated with 10 ng/ml of IL-2. The resulting mNK cells are injected to the patient for autoimmune therapy. Substantial decrease in tumor size is observed for all patients.

What is claimed is:

1. A composition comprising:
a precursor natural killer cell comprising Axl receptor; and
a ligand that is not naturally occurring in the precursor natural killer cell and is configured to form a complex with the Axl receptor, wherein the complex, when formed, is configured to induce the precursor natural killer cell to differentiate into a mature natural killer cell.

2. The composition of claim 1, wherein the ligand is at least one selected from the group consisting of: γ-carboxylated Gas6 protein, γ-carboxylated Gas6 protein homologues, and fragments of γ-carboxylated Gas6 protein, fragments of γ-carboxylated Gas6 protein homologues, and an antibody configured to bind Axl receptor.

3. The composition of claim 1, further comprising a human stromal cell.

4. The composition of claim 1, further comprising a cell configured to express γ-carboxylated Gas6 protein, γ-carboxylated Gas6 protein homologues, or fragments of the foregoing.

5. The composition of claim 4, wherein the cell recited in claim 4 comprises a cell from a cloned cell line.

6. The composition of claim 1, further comprising a mature natural killer cell, wherein the mature natural killer cell is either activated or inactivated to target a cancer cell.

7. The composition of claim 1, further comprising a hematopoietic stem cell.

8. A composition comprising:
a mature natural killer cell comprising an Axl receptor; and
a ligand that is not naturally occurring in the mature natural killer cell, wherein the ligand and the Axl receptor are in the form of a complex.

9. The composition of claim 8, further comprising a precursor natural killer cell.

10. A method to produce the composition of claim 8, comprising:
providing a cell culture;
providing a precursor natural killer cell in the culture, wherein the precursor natural killer cell comprises an Axl receptor; and
providing in the culture a ligand that is not naturally occurring in the precursor natural killer cell such that the ligand and the precursor natural killer cell contact with each other and such that the ligand and the Axl receptor form a complex, wherein the complex, when formed, induces the precursor natural killer cell to differentiate into a mature natural killer cell.

11. The method of claim 10, further comprising separating the mature natural killer cell from the culture.

12. The method of claim 11, further comprising culturing the mature natural killer cell in interleukin-2.

13. The method of claim 12, wherein the concentration of interleukin-2 is from 8 to 15 ng/mL.

14. The method of claim 10, wherein providing the precursor natural killer cell comprises
providing a population of hematopoietic stem cells; and
differentiating at least a part of the population of hematopoietic stem cells into a population comprising precursor natural killer cells.

15. A method to treat a patient in need of mature natural killer cells, comprising:
providing the composition of claim 8; and
administering the composition to the patient in an amount that is effective to treat the patient.

16. The method of claim 15, wherein the composition is administered in an amount that is effective for the treatment of cancer in the patient.

17. A method to treat cancer, comprising:
obtaining a population of hematopoietic stem cells;
differentiating at least a part of the population of hematopoietic stem cells into a population comprising precursor natural killer cells, wherein the precursor natural killer cell comprises an Axl receptor;
contacting at least a part of the population comprising precursor natural killer cells with a ligand that is not naturally occurring in the precursor natural killer cells, thereby differentiating at least part of the precursor natural killer cells into mature natural killer cells; and
administering at least part of the mature natural killer cells to a patient.

18. The method of claim 17, wherein the population of hematopoietic stem cells is obtained from at least one human blood source selected from the group consisting of: bone marrow, peripheral blood, and umbilical cord blood.

19. The method of claim 17, wherein the population of hematopoietic stem cells is obtained from the patient.

20. The method of claim 17, additionally comprising separation of the at least part of the mature natural killer cells from non-mature natural killer cells.

21. The method of claim 17, wherein the ligand is at least one selected from the group consisting of: γ-carboxylated Gas6 protein, γ-carboxylated Gas6 protein homologues, fragments of γ-carboxylated Gas6 protein, fragments of γ-carboxylated Gas6 protein homologues, and an antibody that is configured to bind Axl receptor.


SEQUENCE LISTING (25 sequences)

SEQ ID NO: 1 - DNA, 34 bp, Artifical Sequence
  Synthetic nucleic acid sequence
gtcgacgctcctctcagctgtgatggctaccata

SEQ ID NO: 2 - DNA, 36 bp, Artificial Sequence
  Synthetic nucleic acid sequence
ggatcccagaagacgtctacgggcctcaaattccaa

SEQ ID NO: 3 - DNA, 21 bp, Artificial Sequence
  Synthetic nucleic acid sequence
gtcacgtcgaagtacttggtg

SEQ ID NO: 4 - DNA, 21 bp, Artificial Sequence
  Synthetic nucleic acid sequence
aaccagccacatagcacacat

SEQ ID NO: 5 - DNA, 20 bp, Artificial Sequence
  Synthetic nucleic acid sequence
gtggggcgccccaggcacca

SEQ ID NO: 6 - DNA, 24 bp, Artificial Sequence
  Synthetic nucleic acid sequence
ctccttaatgtcacgcacgatttc

SEQ ID NO: 7 - DNA, 23 bp, Artificial Sequence
  Synthetic nucleic acid sequence
agcggctgactgaactcagattg

SEQ ID NO: 8 - DNA, 22 bp, Artificial Sequence
  Synthetic nucleic acid sequence
gcacagttttcagctctatagg

SEQ ID NO: 9 - DNA, 22 bp, Artificial Sequence
  Synthetic nucleic acid sequence
ccaacatggcctcgccgcagct

SEQ ID NO: 10 - DNA, 22 bp, Artificial Sequence
  Synthetic nucleic acid sequence
ttgtagagaaagcttctggctc

SEQ ID NO: 11 - DNA, 22 bp, Artificial Sequence
  Synthetic nucleic acid sequence
aggtacaaccgcagtaatgcgg

SEQ ID NO: 12 - DNA, 23 bp, Artificial Sequence
  Synthetic nucleic acid sequence
agtgaacattacagatttatccc

SEQ ID NO: 13 - DNA, 21 bp, Artificial Sequence
  Synthetic nucleic acid sequence
gtcacgtcgaagtacttggtg

SEQ ID NO: 14 - DNA, 21 bp, Artificial Sequence
  Synthetic nucleic acid sequence
aaccagccacatagcacacat

SEQ ID NO: 15 - DNA, 29 bp, Artificial Sequence
  Synthetic nucleic acid sequence
ggcctcgagcatgccgccaccgcccgggc

SEQ ID NO: 16 - DNA, 29 bp, Artificial Sequence
  Synthetic nucleic acid sequence
ggcgaattccggtctagggggtggcatgc

SEQ ID NO: 17 - DNA, 29 bp, Artificial Sequence
  Synthetic nucleic acid sequence
ggcctcgagcatgccgccaccgcccgggc

SEQ ID NO: 18 - DNA, 29 bp, Artificial Sequence
  Synthetic nucleic acid sequence
ggcgaattccggtctagggggtggcatgc

SEQ ID NO: 19 - DNA, 20 bp, Artificial Sequence
  Synthetic nucleic acid sequence
ggtgcccatcaacttcggaa

SEQ ID NO: 20 - DNA, 21 bp, Artificial Sequence
  Synthetic nucleic acid sequence
ggatgtcccaggtggaagatt

SEQ ID NO: 21 - DNA, 19 bp, Artificial Sequence
  Synthetic nucleic acid sequence
gtctcccgtacttcctgga

SEQ ID NO: 22 - DNA, 19 bp, Artificial Sequence
  Synthetic nucleic acid sequence
ctcacccactgcaacctgc

SEQ ID NO: 23 - DNA, 19 bp, Artificial Sequence
  Synthetic nucleic acid sequence
agacctacacagtttcctc

SEQ ID NO: 24 - DNA, 21 bp, Artificial Sequence
  Synthetic nucleic acid sequence
ggcccgtggccccttcgctct

SEQ ID NO: 25 - DNA, 20 bp, Artificial Sequence
  Synthetic nucleic acid sequence
ggcctaggctgcggcgggct